(19) United States
(12) Patent Application Publication
(10) Pub. No.: US 2026/0193659 A1
(43) Pub. Date: Jul. 9, 2026

(54) METHODS FOR TREATING MONGE'S DISEASE

(71) Applicant: The Regents of the University of California, Oakland, CA (US)

(72) Inventors: Gabriel G. Haddad, San Diego, CA (US); Priti Azad, La Jolla, CA (US); Dan Zhou, San Marcos, CA (US); Tariq M. Rana, San Diego, CA (US)

(21) Appl. No.: 19/128,790

(22) PCT Filed: Nov. 9, 2023
(86) PCT No.: PCT/US2023/079207

§ 371 Date: May 9, 2025

Related U.S. Application Data
(60) Provisional application No. 63/424,703, filed on Nov. 11, 2022.

(51) Int. Cl. C12N 15/113; A61K 31/4192; A61K 31/4745; A61P 7/00

(52) U.S. Cl. CPC C12N 15/1137; A61K 31/4192; A61K 31/4745; A61P 7/00; C12N 2310/111; C12N 2310/3231; C12N 2310/3233

(57) ABSTRACT

Provided herein are compositions and methods of treating Monge's disease and/or reducing erythrocytosis in a subject that include: administering to the subject a therapeutic agent, wherein the therapeutic agent inhibits expression of a target gene, and wherein the therapeutic agent reduces erythrocytosis. Compositions include CK2 inhibitors and antisense nucleotides targeting IncRNAs.

### CROSS-REFERENCE TO RELATED APPLICATIONS

This application claims priority to U.S. Provisional Patent Application No. 63/424,703, filed on Nov. 11, 2022. The disclosure of the prior application is considered part of the disclosure of this application and is incorporated herein by reference in its entirety.

### FEDERALLY SPONSORED RESEARCH OR DEVELOPMENT

This invention was made with Government support under HL146530 awarded by the National Institutes of Health. The Government has certain rights in the invention.

### SEQUENCE LISTING

This application contains a Sequence Listing that has been submitted electronically as an XML file named “15670-0370WO1.XML.” The XML file, created on Nov. 9, 2023, is 23,026 bytes in size. The material in the XML file is hereby incorporated by reference in its entirety.

### BACKGROUND

Around 140 million people live permanently at high altitude and among them around 5-33% suffer from Monge's disease (Chronic Mountain Sickness, CMS). Excessive erythrocytosis (EE) is a major hallmark of patients suffering from chronic mountain sickness (CMS, Monge's disease) and is responsible for major morbidity and even mortality in early adulthood. The only and inadequate treatment for these patients is periodic phlebotomy and no drug is available that has a major impact on hematocrit levels and can be used as a treatment for reducing excessive erythropoiesis in these individuals. Acetazolamide is the only drug that has been tested for this disease and it showed a mild response and relief of symptoms but does not act as treatment for the excessive production of RBCs.

### SUMMARY

Provided herein are methods of treating Monge's disease in a subject that include: administering to the subject a therapeutic agent, wherein the therapeutic agent inhibits expression of a target gene, and wherein the therapeutic agent reduces erythrocytosis, thereby treating Monge's disease in the subject.

Also provided herein are methods of reducing erythrocytosis in a subject that include: administering to the subject a therapeutic agent, wherein the therapeutic agent inhibits expression of a target gene, thereby reducing erythrocytosis in the subject.

In some embodiments, the therapeutic agent inhibits expression of the target gene by inhibiting a target RNA, wherein the target RNA regulates the expression of the target gene. In some embodiments, the target RNA comprises a long non-coding RNA (lncRNA). In some embodiments, the lncRNA comprises LINC00106, MDC1-AS1, LINC02228, LINC00235, LINC00431, APOBEC3B-AS1, or LINC01133. In some embodiments, the lncRNA comprises LINC02228. In some embodiments, the lncRNA comprises LINC00431. In some embodiments, the lncRNA comprises APOBEC3B-AS1.

In some embodiments, the therapeutic agent comprises an inhibitory nucleic acid. In some embodiments, the inhibitory nucleic acid comprises an antisense oligonucleotide (ASO), a locked nucleic acid (LNA), or a morpholino antisense oligonucleotide. In some embodiments, the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

In some embodiments, the target gene comprises CSNK2B, DXO, ZNRD1, PPP1R11, TAP2, IGF1. VCAM1, SHH, TPO, RHEX, SKIV2L, DAXX. ZBTB12, IER3, or any combinations thereof. In some embodiments, the target gene comprises CSNK2B.

In some embodiments, the therapeutic agent comprises a CK2 inhibitor. In some embodiments, the therapeutic agent comprises TBB or CX-4945. In some embodiments, the therapeutic agent comprises two or more therapeutic agents. In some embodiments, the two or more therapeutic agents comprises the inhibitory nucleic acid, the CK2 inhibitor, or any combinations thereof.

Also provided herein are pharmaceutical compositions comprising a therapeutic agent, wherein the therapeutic agent inhibits expression of a target gene and reduces erythrocytosis.

In some embodiments, the therapeutic agent inhibits expression of the target gene by inhibiting a target RNA, wherein the target RNA regulates the expression of the target gene. In some embodiments, the target RNA comprises a long non-coding RNA (lncRNA). In some embodiments, the lncRNA comprises LINC00106, MDC1-AS1, LINC02228, LINC00235, LINC00431, APOBEC3B-AS1, or LINC01133. In some embodiments, the lncRNA comprises LINC02228. In some embodiments, the lncRNA comprises LINC00431. In some embodiments, the lncRNA comprises APOBEC3B-AS1.

In some embodiments, the therapeutic agent comprises an inhibitory nucleic acid. In some embodiments, the inhibitory nucleic acid comprises an antisense oligonucleotide (ASO), a locked nucleic acid (LNA), or a morpholino antisense oligonucleotide. In some embodiments, the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

In some embodiments, the target gene comprises CSNK2B, DXO, ZNRD1, PPP1R11, TAP2, IGF1, VCAM1, SHH, TPO, RHEX, SKIV2L, DAXX. ZBTB12, IER3, or any combinations thereof. In some embodiments, the target gene comprises CSNK2B. In some embodiments, the therapeutic agent comprises a CK2 inhibitor. In some embodiments, the therapeutic agent comprises TBB or CX-4945. In some embodiments, the therapeutic agent comprises two or more therapeutic agents. In some embodiments, the two or more therapeutic agents comprise the inhibitory nucleic acid, the CK2 inhibitor, or any combinations thereof.

Also provided herein are pharmaceutical compositions comprising a therapeutic agent, wherein the therapeutic agent inhibits expression of a target gene and reduces erythrocytosis, for use in the treatment of Monge's disease.

In some embodiments, the therapeutic agent inhibits expression of the target gene by inhibiting a target RNA, wherein the target RNA regulates the expression of the target gene. In some embodiments, the target RNA comprises a long non-coding RNA (lncRNA). In some embodiments, the lncRNA comprises LINC00106, MDC1-AS1, LINC02228, LINC00235, LINC00431, APOBEC3B-AS1, or LINC01133. In some embodiments, the lncRNA comprises LINC02228. In some embodiments, the lncRNA comprises LINC00431. In some embodiments, the lncRNA comprises APOBEC3B-AS1.

In some embodiments, the therapeutic agent comprises an inhibitory nucleic acid. In some embodiments, the inhibitory nucleic acid comprises an antisense oligonucleotide (ASO), a locked nucleic acid (LNA), or a morpholino antisense oligonucleotide. In some embodiments, the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

In some embodiments, the target gene comprises CSNK2B, DXO, ZNRD1, PPP1R11, TAP2, IGF1. VCAM1, SHH, TPO, RHEX, SKIV2L, DAXX. ZBTB12, IER3, or any combinations thereof. In some embodiments, the target gene comprises CSNK2B. In some embodiments, the therapeutic agent comprises a CK2 inhibitor. In some embodiments, the therapeutic agent comprises TBB or CX-4945. In some embodiments, the therapeutic agent comprises a plurality of therapeutic agents. In some embodiments, the plurality of therapeutic agents comprise the inhibitory nucleic acid, the CK2 inhibitor, or any combinations thereof.

Also provided herein are uses of a pharmaceutical composition comprising a therapeutic agent, in the manufacture of a medicament for treating Monge's disease, wherein the therapeutic agent inhibits expression of a target gene and reduces erythrocytosis.

In some embodiments, the therapeutic agent inhibits expression of the target gene by inhibiting a target RNA, wherein the target RNA regulates the expression of the target gene. In some embodiments, the target RNA comprises a long non-coding RNA (lncRNA). In some embodiments, the lncRNA comprises LINC00106, MDC1-AS1, LINC02228, LINC00235, LINC00431, APOBEC3B-AS1, or LINC01133. In some embodiments, the lncRNA comprises LINC02228. In some embodiments, the lncRNA comprises LINC00431. In some embodiments, the lncRNA comprises APOBEC3B-AS1.

In some embodiments, the therapeutic agent comprises an inhibitory nucleic acid. In some embodiments, the inhibitory nucleic acid comprises an antisense oligonucleotide (ASO), a locked nucleic acid (LNA), or a morpholino antisense oligonucleotide. In some embodiments, the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

In some embodiments, the target gene comprises CSNK2B, DXO, ZNRD1, PPP1R11, TAP2, IGF1, VCAM1, SHH, TPO, RHEX, SKIV2L, DAXX, ZBTB12, IER3, or any combinations thereof. In some embodiments, the target gene comprises CSNK2B. In some embodiments, the therapeutic agent comprises a CK2 inhibitor. In some embodiments, the therapeutic agent comprises TBB or CX-4945. In some embodiments, the therapeutic agent comprises a plurality of therapeutic agents. In some embodiments, the plurality of therapeutic agents comprise the inhibitory nucleic acid, the CK2 inhibitor, or any combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1A shows an exemplary schematic illustration of the experimental strategy. CD34⁺ cells were isolated from blood (PBMCs) obtained from CMS or non-CMS subjects, pooled, and treated with hypoxia or room air (as control). Following the treatment, total RNA was isolated, and the quality was determined with TapeStation. Ribosomedepleted (ribodepleted) libraries were generated and sequenced. The candidate hypoxia-responding lncRNAs were identified and prioritized for further qRT-PCR-based evaluation and functional analyses.

FIG. 1B shows a summary of RNA-Seq results. Hypoxia treatment induced distinct transcriptional responses in CMS and non-CMS cells. A total of 426 or 1,702 hypoxia-induced DEGs were identified in the CMS and non-CMS cells, respectively, with little overlap. Further annotation revealed a distinct group of 5 lncRNAs in the CMS DEGs and 36 lncRNAs in the non-CMS DEGs, suggesting specific lncRNA-mediated hypoxia responses between CMS and non-CMS subjects.

FIG. 2A shows qRT-PCR validation of all 5 lncRNAs that were differentially altered (up- and downregulated) in the CMS cell group. iPSC-derived CD34⁺ cells after exposure to hypoxia and normoxia (for 3 days) were used for this assay. Expression levels were tested and validated in both CMS (n=3) and non-CMS (n=3) cells under hypoxia and normoxia. HIKER/LINC02228 was tremendously upregulated in the CMS cells under hypoxia.

FIG. 2B shows qRT-PCR validation of top 10 upregulated lncRNAs in the non-CMS cell group. iPSC-derived CD34⁺ cells after exposure to hypoxia and normoxia (for 3 days) were used for this assay. Expression levels were tested and validated in both CMS (n=3) and non-CMS (n=3) cells under hypoxia and normoxia.

FIG. 2C shows qRT-PCR validation of top 10 downregulated lncRNAs in the non-CMS cell group. iPSC-derived CD34⁺ cells after exposure to hypoxia and normoxia (for 3 days) were used for this assay. Expression levels were tested and validated in both CMS (n=3) and non-CMS (n=3) cells under hypoxia and normoxia.

FIG. 2D shows nuclear and cytoplasmic localization of lncRNAs. qRT-PCR results of confirmation for the expression changes for HIKER/LINC02228, LINC000431 (nuclear) and LINC01133, and APOBEC3B-AS1 and UBE2Q1-AS1 (cytoplasmic) for CMS, non-CMS and sea-level erythroid cells under hypoxia and normoxia. iPSC-derived CD34⁺ cells after exposure to hypoxia and normoxia (for 3 days) were used for this assay.

FIGS. 3A-3B show qRT-PCR results confirming the expression changes for HIKER/LINC02228 for CMS. non-CMS, and sea-level subjects under normoxia and in response to hypoxia at 5% O2 in iPSC-derived (FIG. 3A) and PBMC-derived native CD34⁺ cells (FIG. 3B).

FIGS. 3C-3D show qRT-PCR results confirming the expression changes for HIKER/LINC02228 for CMS, non-CMS, and sea-level subjects under normoxia and in response to hypoxia at 1% O2 in iPSC-derived (FIG. 3C) and PBMC-derived native CD34⁺ cells (FIG. 3D).

FIG. 3E shows functional analysis of HIKER/LINC02228 in iPSC-derived CD34⁺ cells using methylcellulose colony assay. Panel shows significant reduction of BFU-E under hypoxia with KD of each lncRNA in CMS in each subject.

FIG. 4A shows qRT-PCR results confirming expression changes for RNA-Seq analysis of the KD of HIKER/LINC02228 versus controls. Top 5 upregulated genes are shown. qPCR was performed on iPSC-derived CD34⁺ cells.

FIG. 4B shows qRT-PCR results confirming the expression changes for RNA-Seq analysis of the KD of HIKER/LINC02228 versus controls. Top 5 downregulated genes are shown. qPCR was performed on iPSC-derived CD34⁺ cells.

FIG. 4C shows western blot confirmation of the top 5 downregulated candidates, CSNK2B, DXO, ZNRD1, PP1R11, and TAP2. Week 1 EBs (iPSC derived) were used in this assay as described herein. Left: representative image for each protein candidate. Right: summary of densitometric analysis of each protein with n=3 for each group.

FIG. 4D shows functional analysis of HIKER/LINC02228 as well as CSNK2B-OE-LINC02228-KD in iPSC-derived CD34+ cells using methylcellulose colony assay. With the OE of CSNK2B gene in the background of HIKER/LINC02228 KD, mean number of BFU-E colonies/CD34⁺ is increased, suggesting a critical function of this gene in the mechanism of action of HIKER/LINC02228.

FIG. 5A shows CSNK2B KD in CMS decreases BFU-E, and CSNK2B OE in non-CMS increases BFU-E, suggesting its critical role in regulating erythropoiesis.

FIG. 5B shows effect of CK2 inhibitor on CMS cells. TBB decreases BFU-E colonies in CMS cells in a dose-dependent manner.

FIG. 5C shows effect of CK2 inhibitor on CMS cells. CX4945 decreases BFU-E colonies more drastically in the CMS cells in a dose-response manner.

FIG. 5D shows CSNK2B KD results in major expression changes of critical TFs. qPCR results confirm decreased expression of TAL1, KLF1, RUNX1. IKAROS, and GATA1.

FIG. 5E is a graph showing GATA1 expression as measured by qPCR in CMS cells, CMS cells with CSNK2B KD, CMS cells treated with CK2 inhibitor, non-CMS cells, and non-CMS cells with CSNK2B-OE. GATA1 expression levels were altered significantly by modulation of CSNK2B levels in CMS and non-CMS cells under hypoxia.

FIG. 5F is a graph showing the effect of CSNK2B and GATA1 modulation on colony-forming potential of CMS and non-CMS cells. GATA1 OE partially rescues the erythropoietic suppression caused by CSNK2B in CMS. Further, KD of GATA1 in non-CMS results in loss of excessive erythropoiesis caused by OE of CSNK2B.

FIG. 6A shows representative images of hemoglobin signal in control and csnk2b morphants stained with o-dianisidine at 2 dpf. Images shown are ventral views with heads to the top. The results show that Csnk2b is required for hemoglobinization of zebrafish erythrocytes.

FIG. 6B shows statistical analyses of dose-dependent loss of hemoglobin in embryos injected with 1, 3, or 5 ng of control (CTL) or csnk2b morpholino.

FIG. 6C shows statistical analysis of hemoglobin phenotypes in control and csnk2b morphants with or without rescue of csnk2b mRNA. Representative images of hemoglobin classification criterion are shown on the right side of the graph. Data collected from 3 independent experiments, with corresponding embryo numbers displayed on the columns.

FIG. 7A shows KD efficiency of LINC02228 (HIKER) by ASO-1. ASO-2, ASO-3 in CMS cells determined by qPCR. All 3 types of ASO tested exhibited >80% KD efficiency. ASO-1 with the highest KD efficiency was used in subsequent experiments.

FIG. 7B shows KD or OE efficiency of CSNK2B in CMS or non-CMS cells determined by qPCR.

FIG. 7C shows KD or OE efficiency of GATA1 in CMS or non-CMS cells determined by qPCR.

FIGS. 7D-F show the mRNA expression levels (qPCR) of the constructs, including LINC02228 (HIKER, FIG. 7D). CSNK2B (FIG. 7E), and GATA1 (FIG. 7F), used in colony forming assays.

FIG. 8 shows functional analysis of BFU colony forming assay with LINC02228-KD or LINC00431-KD CMS cells.

FIG. 9 shows protein sequence similarity between human and zebrafish CSNK2B.

FIGS. 10A-10B show CSNK2B mRNA expression levels (qPCR) in PBMC-derived native CD34+ (FIG. 10A) and iPSC-derived CD34+ (FIG. 10B) at 5% O2.

FIGS. 10C-10D show CSNK2B mRNA expression levels (qPCR) in PBMC-derived native CD34+ (FIG. 10C) and iPSC-derived CD34+ (FIG. 10D) at 1% O2.

FIGS. 11A-11B show LINC00431 plays an important role in erythroid development, specifically in later erythroid stages (CFU) (FIG. 11A) and reticulocyte stage (FIG. 11B).

FIG. 11C shows that LINC00431 plays an important role in reticulocyte stage in both CMS and non-CMS cells.

FIG. 12A shows the validation of LINC00431 target genes by qPCR. Only IER3 showed significant changes.

FIG. 12B shows the functional validation of IER3 as an important target of LINC00431 by erythroid cell culture.

FIG. 13A shows the chromosomal position and composition of the APOBEC3 gene family.

FIG. 13B shows the expression changes (qPCR) of the APOBEC genes with OE of APOBEC3B-AS1 in non-CMS cells.

FIG. 14A shows APOBEC3B mRNA stabilization regulated by APOBEC3B-AS1 in response to actinomycin treatment.

FIG. 14B shows BFU-E colony forming assay conducted to test the functional interaction of APOBEC3B with APOBEC3B-AS1. KD of APOBEC3B significantly reduced the erythropoietic response caused by OE of APOBEC3B-AS1, suggesting a functional role of this gene in these erythroid cells.

### DETAILED DESCRIPTION

Excessive erythrocytosis (EE) is a major hallmark of patients suffering from chronic mountain sickness (CMS, also known as Monge's disease) and is responsible for major morbidity and even mortality in early adulthood. By using RNA-seq as well as downstream functional in-vitro assays in human cells from unique populations, e.g., one living at high altitude showing EE, with another population, at the same altitude and region, showing no evidence of EE (non-CMS), a unique profile of long noncoding RNAs (lncRNAs) in the patients (CMS) as well as adapted group (non-CMS), and critical downstream targets of lncRNA such as CSNK2B (regulatory subunit of CK2) that can regulate erythropoiesis was discovered. Furthermore, usage and testing of inhibitors of CK2 (e.g., TBB and Silmitasertib) curb excessive erythropoiesis (e.g., 50-75% reduction in BFU colonies) in the cells of the CMS patients. These discoveries open an avenue for developing therapy as well as the possibility of screening for CMS and non-CMS subjects in high altitudes. These discoveries could possibly be translated to other patients at sea level with disturbances in erythropoiesis.

Provided herein are methods of treating Monge's disease and/or reducing erythrocytosis in a subject that include: administering to the subject a therapeutic agent, wherein the therapeutic agent inhibits expression of a target gene, and wherein the therapeutic agent reduces erythrocytosis.

Also provided herein are pharmaceutical compositions comprising a therapeutic agent, wherein the therapeutic agent inhibits expression of a target gene and reduces erythrocytosis. In some embodiments, the pharmaceutical composition is used in the treatment of Monge's disease.

Also provided herein are uses of pharmaceutical composition comprising a therapeutic agent, in the manufacture of a medicament for treating Monge's disease, wherein the therapeutic agent inhibits expression of a target gene and reduces erythrocytosis.

Various non-limiting aspects of these methods are described herein and can be used in any combination without limitation. Additional aspects of various components of the methods described herein are known in the art.

It is noted that as used in the specification and the appended claims, the singular forms “a”, “an”, and “the” refer to one or more (i.e., at least one) of the grammatical object of the article unless the context clearly dictates otherwise. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein. “nucleic acid” or “nucleic acid molecule” is used to include any compound and/or substance that comprise a polymer of nucleotides. In some embodiments, a polymer of nucleotides is referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. The terms “polynucleotides,” “nucleic acid,” and “oligonucleotides” can be used interchangeably. They can refer to a polymeric form of nucleotides of any length, either deoxyribonucleotides or ribonucleotides, or analogs thereof polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

### Methods of Treating Monge's Disease

Provided herein are methods of treating Monge's disease and/or reducing erythrocytosis in a subject (e.g., humans) that include administering to the subject a therapeutic agent to inhibit expression of a target gene, and wherein the therapeutic agent reduces erythrocytosis.

Also provided herein are methods of reducing or treating erythrocytosis and/or polycythemia in a subject (e.g., humans) that include administering to the subject a therapeutic agent to inhibit expression of a target gene, thereby reducing or treating erythrocytosis and/or polycythemia. Also provided herein are methods of reducing the concentration of red blood cells in the blood of a subject (e.g., human) that include administering to the subject a therapeutic agent to inhibit expression of a target gene, and wherein the therapeutic agent reduces erythrocytosis. Also provided herein are methods of preventing chronic mountain sickness symptoms in a subject (e.g., human) that include administering to the subject a therapeutic agent to inhibit expression of a target gene, and wherein the therapeutic agent reduces erythrocytosis. Also provided herein are methods of reducing hematocrit and/or hemoglobin levels in the blood of a subject (e.g., human) that include administering to the subject a therapeutic agent to inhibit expression of a target gene, and wherein the therapeutic agent reduces erythrocytosis. Also provided herein are methods of reducing a number of erythroid colonies in the blood of a subject (e.g., human) that include administering to the subject a therapeutic agent to inhibit expression of a target gene, and wherein the therapeutic agent reduces erythrocytosis.

### Monge's Disease or Chronic Mountain Sickness (CMS)

Monge's disease or chronic mountain sickness (CMS) refers to a clinical syndrome caused by a subject's chronic exposure to high-altitude hypoxia, wherein the proportion of blood volume that is occupied by red blood cells increases (polycythaemia) and there is an abnormally low level of oxygen in the blood (hypoxemia). CMS typically develops after extended time living at high altitude (e.g., over 2,500 metres (8,200 ft)). The main feature of CMS is excessive erythrocytosis (EE) that exhibits high hematocrit/Hb levels in blood, and it is most common amongst native populations of high-altitude nations. This excessive pathobiological response to hypoxia has deleterious effects, since a high hematocrit/hemoglobin increases blood viscosity and reduces blood flow to hypoxia-sensitive organs (e.g., brain and heart), often resulting in myocardial infarction, stroke, and high mortality in young adults. The most frequent symptoms of CMS are headache, dizziness, tinnitus, breathlessness, palpitations, sleep disturbance, fatigue, loss of appetite, confusion, cyanosis, and dilation of veins.

As used herein, the term “non-CMS subject” refers to an individual who live at the same geographic location and altitude as a subject with CMS (“CMS subject”) but are adapted and do not show any of the traits of the CMS individual.

Hemoglobin (Hb) is a protein contained in red blood cells that is responsible for delivery of oxygen to the tissues. The amount of hemoglobin in whole blood can be expressed in grams per deciliter (g/dl), wherein a normal Hb level for a male can be about 14 to about 18 g/dl (e.g., about 14 to about 17, about 14 to about 16, about 14 to about 15, about 15 to about 18, about 15 to about 17, about 15 to about 16, about 16 to about 18, about 16 to about 17, or about 17 to about 18 g/dl), and that for a female can be about 12 to about 16 g/dl (e.g., about 12 to about 15, about 12 to about 14, about 12 to about 13, about 13 to about 16, about 13 to about 15, about 13 to about 14, about 14 to about 16, about 14 to about 15, or about 15 to about 16 g/dl). Hematocrit level refers to the percentage of red blood cells in the blood of a subject, wherein the hematocrit level can be measured by comparing the volume of red blood cells to the total blood volume (red blood cells and plasma). In some embodiments, the normal hematocrit level for a male is about 40 to about 54% (e.g., about 40 to about 52, about 40 to about 50, about 40 to about 48, about 40 to about 46, about 40 to about 44, about 40 to about 42, about 42 to about 54, about 42 to about 52, about 42 to about 50, about 42 to about 48, about 42 to about 46, about 42 to about 44, about 44 to about 54, about 44 to about 52, about 44 to about 50, about 44 to about 48, about 44 to about 46, about 46 to about 54, about 46 to about 52, about 46 to about 50, about 46 to about 48, about 48 to about 54, about 48 to about 52, about 48 to about 50, about 50 to about 54, about 50 to about 52, or about 52 to about 54%), and for a female it is about 36 to about 48% (e.g., about 36 to about 46, about 36 to about 44, about 36 to about 42, about 36 to about 40, about 36 to about 38, about 38 to about 48, about 38 to about 46, about 38 to about 44, about 38 to about 42, about 38 to about 40, about 40 to about 48, about 40 to about 46, about 40 to about 44, about 40 to about 42, about 42 to about 48, about 42 to about 46, about 42 to about 44, about 44 to about 48, about 44 to about 46, or about 46 to about 48%). See, e.g., Billett, Clinical Methods: The History, Physical, and Laboratory Examinations. 3rd edition. PMID: 21250102. 1990, which is herein incorporated by reference in its entirety.

As used herein, the term “erythrocytosis” or “polycythemia” refers a high concentration of red blood cells in the blood of a subject, especially resulting from a known stimulus (e.g., hypoxia). In some embodiments, excessive erythrocytosis (EE) refers to a condition with Hb≥21 g/dL in men, Hb≥19 g/dL in women. Erythropoiesis refers to a process which produces red blood cells (erythrocytes), which includes the development from hematopoietic stem cell to mature red blood cell, wherein erythroid cells differentiate from hematopoietic stem cells (HSC) in the bone marrow. The long-term HSCs successively differentiate into the multipotent progenitors CLP (common lymphoid progenitors) and CMPs (common myeloid progenitors); the CMPs differentiate to GMPs (granulocyte monocyte precursors), or MEPs (megakaryocyte/erythroid precursors); and MEPs differentiate into committed BFU-Es (blast-forming unit-erythroid cells), which then give rise to CFU-Es (colony-forming unit-erythroid cells). The CFU-Es then mature along various intermediate stages, wherein the final maturation stage is the generation of reticulocytes, which then enucleate and are released into the blood stream as red blood cells (RBCs). See e.g., Fan et al., Epigenetic Gene Expression and Regulation. 2015, doi.org/10.1016/C2013-0-14005-6, which is herein incorporated by reference in its entirety. In some embodiments, the methods herein can be used to treat or prevent erythrocytosis or polycythemia.

### Therapeutic Agents Reducing Erythrocytosis

In some embodiments, any one of the methods described herein includes administering to a subject a therapeutic agent, wherein the therapeutic agent reduces erythrocytosis in the subject. In some embodiments, reducing erythrocytosis can include reducing the number of red blood cells in the blood of a subject. In some embodiments, reducing erythrocytosis can include lowering red blood cell production in a subject. In some embodiments, reducing erythrocytosis can include reducing the concentration of red blood cells in the blood in a subject.

In some embodiments, any one of the methods described herein includes administering to a subject a therapeutic agent, wherein the therapeutic agent inhibits expression of a target gene, wherein the target gene regulates erythropoiesis in the subject. In some embodiments, any one of the methods described herein includes administering a therapeutic agent that inhibits expression of the target gene by inhibiting a target RNA, wherein the target RNA regulates the expression of the target gene. In some embodiments, the target RNA comprises a long non-coding RNA (lncRNA). As used herein, a “long noncoding RNA (lncRNA)” refers to a transcript that has more than 200 nucleotides and is not translated into protein or has no or limited coding capacity. In some embodiments, lncRNAs include intergenic lincRNAs, intronic ncRNAs, and sense and antisense lncRNAs. In some embodiments, lncRNAs can regulate gene specific transcription. In some embodiments, lncRNAs can regulate post-transcriptional mRNA processing, e.g., mRNA splicing, protein translation, or siRNA-directed gene regulation. In some embodiments, lncRNAs can regulate epigenetic modifications, including histone and DNA methylation, histone acetylation and sumoylation, that affect many aspects of chromosomal biology. In some embodiments, lncRNAs can regulate DNA replication timing and chromosome stability.

In some embodiments, a target RNA can include a lncRNA, wherein the lncRNA regulates erythropoiesis. In some embodiments, a lncRNA comprises LINC00106, MDC1-AS1, LINC02228, LINC00235, LINC00431, APOBEC3B-AS1, or LINC01133. In some embodiments, a lncRNA comprises LINC02228. In some embodiments, a lncRNA comprises LINC00431. In some embodiments, a lncRNA comprises APOBEC3B-AS1.

In some embodiments, a therapeutic agent inhibits expression of a target gene by inhibiting a target RNA, wherein the target RNA regulates the expression of the target gene. In some embodiments, a therapeutic agent comprises an inhibitor of the target RNA, wherein the target RNA comprises a lncRNA. In some embodiments, a therapeutic agent comprises an inhibitor of the target gene. In some embodiments, a therapeutic agent comprises a inhibitory protein, inhibitory oligonucleotide, or any combinations thereof.

### Inhibitory Nucleic Acids

In some embodiments, a therapeutic agent comprises an inhibitory nucleic acid. Inhibitory nucleic acids in any of the methods and compositions described herein can include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of a target RNA and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or any combinations thereof see, e.g., WO 2010040112, which is herein incorporated by reference in its entirety. In some embodiments, the inhibitory nucleic acid inhibits the target RNA by knockdown of the target RNA expression.

In some embodiments, an inhibitory nucleic acid can be 10 to 50 (e.g., 10 to 40, 10 to 35, 10 to 30, 10 to 20, 20 to 50, 20 to 40, 20 to 30, 30 to 50, 30 to 40, or 40 to 50) nucleotides in length. In some embodiments, an inhibitory nucleic acid can have a complementary portion of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin.

In some embodiments, an inhibitory nucleic acid is sufficiently complementary to the target RNA, i.e., hybridize sufficiently well and with sufficient specificity, to give the desired effect. As used herein, “complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a target RNA, then the bases are considered to be complementary to each other at that position. In some embodiments, 100% complementarity is not required. In some embodiments, an inhibitory nucleic acid described herein can have at least 80% sequence complementarity to a target region within the target RNA, e.g., 90%, 95%, or 100% sequence complementarity to the target region within the target RNA.

For further disclosure regarding inhibitory nucleic acids, see, e.g., US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids), which are herein incorporated by reference in their entireties.

In some embodiments, the inhibitory nucleic acid comprises an antisense oligonucleotide (ASO), a locked nucleic acid (LNA), or a morpholino antisense oligonucleotide.

In some embodiments, the inhibitory nucleic acid comprises an antisense oligonucleotide (ASO). Antisense oligonucleotide (ASO) refers to single-stranded chains of synthetic nucleic acids that are complementary to target RNA. Antisense oligonucleotides are typically designed to block expression of a DNA or RNA target by binding to the target and halting expression at the level of transcription, translation, or splicing. In some embodiments, ASOs can be used for knocking down gene functions. In some embodiments, the inhibitory nucleic acid comprises an ASO, wherein the ASO comprises any one of SEQ ID NOs:1-6.

In some embodiments, the inhibitory nucleic acid comprises a locked nucleic acid (LNA). As used herein, locked nucleic acids (LNAs), also known as bridged nucleic acid (BNA), and often referred to as inaccessible RNA, is a modified RNA nucleotide in which the ribose moiety is modified with an extra bridge connecting the 2′ oxygen and 4′ carbon.

The bridge “locks” the ribose in the 3′-endo (North) conformation, wherein this structure provides for increased stability against enzymatic degradation. LNAs also have increased affinity to base pair with RNA as compared to DNA. In some embodiments, these properties render LNAs especially useful as probes for fluorescence in situ hybridization (FISH) and comparative genomic hybridization, as knockdown tools for miRNAs, and as antisense oligonucleotides to target RNAs as described herein. In some embodiments, an LNA molecule can include molecules comprising 10-30 (e.g., 12, 13, 14, 15, 16, 17, 18, 19, 20, 21, 22, 23, 24, 25, 26, 27, 28, 29, or 30) nucleotides in each strand, wherein one of the strands is substantially identical, e.g., at least 80% (or more. e.g., 85%, 90%, 95%, or 100%) identical, e.g., having 3, 2, 1, or 0 mismatched nucleotide(s), to a target region in the target RNA. The LNA molecules can be chemically synthesized using methods known in the art.

In some embodiments, an LNA can target a lncRNA, wherein the lncRNA comprises LINC02228 or LINC00431.

In some embodiments, the inhibitory nucleic acid comprises a morpholino antisense oligonucleotide. A morpholino antisense oligonucleotide is a type of oligomer nucleic acid with a molecular structure containing DNA bases attached to a backbone of methylenemorpholine rings linked through phosphorodiamidate groups. In some embodiments, morpholino antisense oligonucleotides are used as research tools for reverse genetics by knocking down gene function. In some embodiments, a morpholino antisense oligonucleotide can block the translation initiation of gene CSNK2B. In some embodiments, a morpholino antisense oligonucleotide can comprise SEQ ID NO: 23.

### Inhibitory Compounds

In some embodiments, any one of the methods described herein includes a therapeutic agent that inhibit expression of a target gene. In some embodiments, the target gene is located in a DNA-selected region that plays a role in Monge's disease. In some embodiments, the target gene is a differentially expressed gene between a CMS cell and a non-CMS cell. In some embodiments, the target gene is upregulated when knockdown (KD) of a target RNA (e.g., lncRNA) occurs. In some embodiments, the target gene is downregulated when knockdown (KD) of a target RNA (e.g., lncRNA) occurs. In some embodiments, the target gene comprises CSNK2B, DXO, ZNRD1, PPP1R11, TAP2, IGF1, VCAM1, SHH, TPO, RHEX, SKIV2L, DAXX, ZBTB12, IER3, or any combination thereof. In some embodiments, the target gene comprises CSNK2B.

In some embodiments, a therapeutic agent comprises an inhibitor of a target gene. In some embodiments, a therapeutic agent comprises a CK2 inhibitor. In some embodiments, the therapeutic agent comprises CX-4945 (silmitasertib), CX-5011, compound 9e, GO289, CIGB-300, DBC, Fisetin, compound 8h, Emodin, TBI (K17), CK2-IN-9, or TBB. In some embodiments, the therapeutic agent comprises TBB or CX-4945. In some embodiments, the therapeutic agent comprises CX-4945 (silmitasertib), wherein CX-4945 is an oral drug used and FDA approved for other diseases (e.g., Advanced Basel Cell Carcinoma, Cholangiocarcinoma). In some embodiments, the therapeutic agent comprises CX-4945, wherein CX-4945 is used for a treatment for Monge's disease, erythrocytosis, or polycythemia.

### Pharmaceutical Compositions

Provided herein are pharmaceutical compositions comprising a therapeutic agent, wherein the therapeutic agent inhibits expression of a target gene and reduces erythrocytosis. In some embodiments, a pharmaceutical composition comprises a therapeutic agent that inhibits expression of a target gene and reduces erythrocytosis, for use in the treatment of Monge's disease. Also provided herein are uses of a pharmaceutical composition comprising a therapeutic agent, in the manufacture of a medicament for treating Monge's disease, wherein the therapeutic agent inhibits expression of a target gene and reduces erythrocytosis.

The methods described herein can include the administration of pharmaceutical compositions and formulations comprising inhibitory nucleic acid sequences designed to target lncRNAs or target genes that regulate erythropoiesis.

In some embodiments, the pharmaceutical compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the pharmaceutical compositions described herein include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants: cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences as described herein) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical as described herein and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl. and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations can be administered for prophylactic and/or therapeutic treatments. In some embodiments, for therapeutic applications, compositions are administered to a subject who is at risk of or has a disorder described herein (e.g., Monge's disease), in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age, and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69: Johnson (1995) J. Pharm. Sci. 84:1144-1146: Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as described herein are correct and appropriate.

Single or multiple administrations of formulations can be given depending on for example: the dosage and frequency as required and tolerated by the patient, the degree and amount of therapeutic effect generated after each administration, and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray, or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT 2/4 35) for all doses with the exception of the 75 mg/kg dose of miR-122 ASO, which showed a very mild increase in ALT and AST levels. They concluded that 50 mg/kg was an effective, non-toxic dose. Another study by Krützfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

### EXAMPLES

The disclosure is further described in the following examples, which do not limit the scope of the disclosure.

### A. LINC002228 and CSNK2B

### Patient Samples

All subjects used in this study (CMS and non-CMS) were adult males, lifelong residents of Cerro de Pasco, Peru, and living at an elevation of approximately 4,338 m. CMS patients fulfilled the diagnostic criteria for CMS, or Monge's disease, based on hematocrit, O₂ saturation, and CMS score. Sea-level individuals used in this study are individuals who have permanently resided at sea level and are within the age group of CMS and non-CMS subjects. Native CD34⁺-derived erythroid cells. Blood samples for PBMC isolation were obtained in sodium heparin-coated tubes. PBMCs were isolated using Histopaque 1077 (Sigma-Aldrich, 10771) by gradient centrifugation. The Dynabeads CD34+Isolation Kit (Invitrogen, 11301D) was used to purify the CD34⁺ fraction. CD34⁺ cells were expanded for a week (days 0-7) in StemSpan medium (STEMCELL Technologies, 09600) containing hydrocortisone (MilliporeSigma, H6909), 50 ng/mL SCF (Peprotech. 300-07), 50 ng/mL FLT3L (Peprotech, 300-19), 10 ng/mL IL-3 (Peprotech, 200-03), 1 ng/mL BMP4 (Peprotech, 120-05), 40 ng/mL IL-11 (Peprotech, 200-11), and 2 U/mL EPO (Amgen, 55513014810). After expansion, cells were further differentiated. Briefly, cells were then cultured in erythroid differentiation medium (EDM), which includes IMDM supplemented with stabilized glutamine (MilliporeSigma. FG0465), 330 μg/mL holo-human transferrin (MilliporeSigma, T0665), 10 μg/mL recombinant human insulin (MilliporeSigma, I9278), 2 IU/mL heparin, and 5% plasma (Innovative Research, IPLAWBCPD).

iPSC-Derived Erythroid Cells

The iPSC lines from CMS, non-CMS, and sea-level subjects have been generated and well characterized. The iPSCs were thoroughly assessed using various methods, including DNA fingerprinting, high-resolution karyotyping, and alkaline phosphatase staining, as well as the expression of multilineage differentiation markers. The erythroid cultures were generated from iPSCs, and the characteristics of these generated erythroid cells of CMS and non-CMS subjects have been studied in detail, including the cluster of differentiation (CD) markers, maturation, and hemoglobin. Briefly, the erythroid cultures were started with approximately 10⁷ to 10⁸ cells of human iPSC cell lines in all subjects. Human iPSCs were differentiated from erythroid cells by formation of embryoid bodies (EBs) for 27 days in a liquid culture medium with the base medium IMDM (MilliporeSigma, FG0465) along with 450 μg/mL holo human transferrin (MilliporeSigma, T0665), 10 μg/mL recombinant human insulin (MilliporeSigma, I9278), 2 IU/mL heparin (NDC 63739-920-25 purchased from McKesson), and 5% human plasma (Innovative Research, IPLAWBCPD) in the presence of 100 ng/mL SCF (Peprotech, 300-07), 100 ng/mL TPO (Peprotech, 300-18), 100 ng/mL FLT3 ligand (Peprotech, 300-19), 10 ng/mL rhu bone morphogenetic protein 4 (BMP4) (Peprotech, 120-05), 5 ng/mL rhu VEGF (Peprotech, 100-20), 5 ng/mL IL-3 (Peprotech, 200-03), 5 ng/mL IL-6 (PeproTech, 200-06), and 3 U/mL Epo (Amgen, 55513014810, purchased from McKesson). This was followed by terminal differentiation as single cells with base medium IMDM (Millipore Sigma. FG0465) along with 5% human plasma (Innovative Research, IPLAWBCPD), 2 IU/mL heparin (McKesson. NDC 63739-920-25), 100 ng/mL SCF (Peprotech, 300-07), 5 ng/mL IL-3 (Peprotech, 200-03), and 3 IU/mL EPO (Amgen, 55513014810).

### RNA-Seq and Data Analysis

Native CD34⁺ cells were isolated from PBMCs as described herein to determine differentially expressed lncRNAs. To do so, RNA was isolated from the erythroid cells after 3 days of exposure to hypoxia or normoxia in CMS (n=4) and nonCMS (n=2). RNA was isolated using the Zymo RNA Kit (Zymo. R1050) per the manufacturer's instructions. The quality of RNA was assessed using TapeStation (Agilent). Ribosome depletion-prepared CMS or non-CMS samples were balanced pooled, and the sequencing libraries were generated by using the TruSeq Stranded Total RNA with RiboZero Gold Library Preparation Kit (Illumina, RS-122-2301). The ribosome-depleted prepared libraries were sequenced using the HiSeq 2500 System in Rapid Run mode (Illumina). A total number of approximately 50 million reads per library were obtained. The resulting reads were mapped using the RUM alignment package with default setting to the human reference hg38. The aligned reads were then processed with htseq-count to obtain the number of reads mapped to genes (Illumina's iGenome GTF annotation for hg38). Quality control (QC) processes were performed prior to and after alignment to ensure high quality of final results. This included GC content, the presence of adaptors, FastQC (www.bioinformatics.babraham.ac.uk/projects/fastqc/) for sequence quality, overrepresented k-mers, and duplicated reads, and Picard (broadinstitute.github.io/picard/)/RseQC for mapping quality. Differentially expressed transcripts were determined by EBSeq. LNCipedia (lncipedia.org/) and GENCODE (www.gencodegenes.org/) were used for lncRNA annotation.

To determine DEGs following HIKER/LINC02228-KD or LINC00431-KD, total RNA was isolated from the CMS iPSC-derived CD34+ with or without a KD of HIKER/LINC02228 or LINC00431 using the Zymo RNA Kit (Zymo, R1050), and the RNA-Seq libraries were generated using the Illumina TruSeq Stranded Total RNA Kit (Illumina, catalog RS-122-2301) per the manufacturer's instructions. A total of more than 40 million reads per library were obtained following sequencing with the HiSeq 2500 System. After QC, the resulting reads were mapped using the RUM alignment package with default setting to the human reference hg38. Differentially expressed transcripts were determined by DESeq2.

Cellular Fractionation and qPCR Analysis of Differentially Expressed lncRNAs

Briefly, total nuclear and cytoplasmic extracts were isolated from erythroid cultures (iPSC-derived CD34⁺ cells isolated from EBs) using Active Motif (catalog 40010) according to the manufacturer's instructions. qPCR for HIKER/LINC02228, LINC01133, APOBEC3B-AS1, UBE2Q-AS1, and LINC00431 were used to assess the purity of the fractions. Primers are listed in Table 1.

KD of Nuclear lncRNA HIKER LINC02228 and LINC00431 Expression Using QIAGEN LNA Gapmers ASO

Locked nucleic acids (LNAs) targeting HIKER/LINC02228 and LINC00431 were designed and synthesized by Exiqon. Detailed sequences are listed in Table 1. The most efficient ASO for each LNA was initially tested in the pilot experiment with and without transfection reagent (Lipofectamine 3000. Life Technologies. L3000-008) in a dose-response experiment at a concentration of 10 nM, 25 nM, 50 nM, and 100 nM. The uptake and the effect of ASO were monitored by qPCR at various stages (iPSC stage and CD34+ cells isolated from EBs). For both lncRNAs, the optimal delivery for all the stages was at the 50 nM concentration without the transfection reagent.

TABLE 1
List of Oligonucleotides and primers for qPCR used in the study.

| | Oligonucleotides and Primers | |
|---|---|---|
| Name | Sequence (5′→3′) | SEQ ID NO |
| LINC02228-ASO1 | G*G*T*T*G*C*T*T*T*C*A*G*G*C*C*C*C*T*T*A | 1 |
| LINC02228-ASO2 | C*T*T*C*A*C*C*A*T*G*C*T*T*G*T*G*C*T*C*T | 2 |
| LINC02228-ASO3 | G*C*C*T*T*C*C*G*G*G*C*C*G*T*C*C*A*C*G*C | 3 |
| LINC00431-ASO1 | T*T*T*C*C*T*C*G*G*T*C*C*A*T*G*C*A*C*A*C | 4 |
| LINC00431-ASO2 | A*C*G*A*T*A*C*C*C*T*T*G*C*T*C*C*C*A*A*C | 5 |
| LINC00431-ASO3 | A*C*C*G*G*C*T*C*C*C*A*T*G*G*T*G*T*C*T*T | 6 |
| LINC01133-L | GGAGCGAGATCCCTCCAAAAT | 7 |
| LINC01133-R | GGCTGTTGTCATACTTCTCATGG | 8 |
| GAPDH-L | CCACATCGCTCAGACACCAT | 9 |
| GAPDH-R | ACCAGGCGCCCAATACG | 10 |
| MDC1-AS-L | TCCCAGATGTGCCAAAGTCAG | 11 |
| MDC1-AS-R | AGCAACCCCAGTTGTCATTC | 12 |
| RAB11B-AS1-L | GGAACATGTTTACATGGACTTTGT | 13 |
| RAB11B-AS1-R | TCTTTGTTCTTGTTTGTTTTCTTTCT | 14 |
| LINC02228-L | CTGAAAGCAACCTCCAGTCC | 15 |
| LINC02228-R | GCCAGTTGCATATTGCTTCA | 16 |
| APOBEC3B-AS1-L | CTCTTACTGCTGGGCCTGTC | 17 |
| APOBEC3B-AS1-R | AGACCGTTCACCACCATCTC | 18 |
| LINC00674-L | CATTGCTGAAGTTGGACTCG | 19 |
| LINC00674-R | TCTCGCAGGCTAAACTGACC | 20 |
| SIAH2-AS1-L | GTTTCCTCCCTCCTCAATCC | 21 |
| SIAH2-AS1-R | CCTCTGCAGACGTGTATTCG | 22 |

Isolation of CD34⁺ Cells from WPSC-Derived EBs.

CD34⁺ cells were isolated from iPSC-derived EBs as follows. After 7 days of differentiation, EBs were harvested by spinning at 400 g for 10 minutes. After centrifugation, EBs were dissociated into single cells using Accutase treatment for 10 minutes and then filtered through a 60 μm cell strainer (Falcon). CD34⁺ cells were isolated from this cell suspension using EasySep Human CD34 Positive Selection Kit 11 (STEMCELL Technologies, 17856) per the manufacturer's instructions. These iPSC-derived CD34+ cells were used in subsequent qPCR and colony-forming assays.

### BFU-E and CFU-E Assays

CD34⁺ cells used in this assay were derived from iPSC-generated EBs as described herein. CD34⁺ cells were plated at a density of 10⁵ cells per 35 mm dish combined with MethoCult H4034 Optimum Media (STEMCELL Technologies, 04044) and 2% FBS. Dishes were incubated at 37° C. in an incubator with 5% CO2 and 5% O2 for 14 days, at which time colonies were scored for BFU-E and CFU-granulocyte, erythrocyte, monocyte, megakaryocyte (CFU-GEMM).

### KD and OE Constructs for CSNK2B and Lentiviral Transduction

KD lentiviral particles were purchased from Santa Cruz Biotechnology Inc., and OE construct and lentiviral particles were generated by Vector Builder. The iPSCs from CMS and non-CMS cells were transduced with polybrene (8 μg/mL, MilliporeSigma, TR-1003-G) at MOI within the range of 1 to 5 (with the titer of lentivirus ranging from 10′ to 109). The optimal concentration was determined for the transduction and antibiotic selection by performing dose-specific kill curves. Transduced cells were selected at 0.5 μg/mL puromycin (Sigma-Aldrich, 58-58-2) or 0.5 μg/mL blasticidin (EMD Millipore, 20-335). For double KD, puromycin and blasticidin combinations were used for selection. The expression of CSNK2B in each construct was verified by qPCR at the iPSC stage as well as the iPSC-derived CD34⁺ stage.

### In Vitro Casein Kinase Inhibitor Experiments

TBB (catalog ab120988) and CX4945 (catalog S2248) were purchased from Abcam and Selleckcam, respectively. Dose-response experiments were performed with the inhibitors using the following concentrations in the colony forming assays using iPSC-derived CD34⁺ cells as described herein: TBB (25 μM, 50 M, and 100 μM) and CX4945 (2.5 μM, 5 μM, and 10 μM).

### Western Blot Analysis for Quantification of Protein Levels.

Proteins were isolated using standard protein isolation protocols with RIPA buffer (Cell Signaling Technology. 9806) and protease inhibitor cocktail (Roche, 11697498001). For protein isolation, EBs at week 1 were used in this study. Through FACS analysis, it is determined that at this stage, the population of erythroid cells was at the CD34⁺ stage. Antibodies against CSNK2B (Abcam, catalog ab76025), DXO (Abcam, catalog ab152135), PPP1R11 (Abcam, catalog ab171960), ZNRD1 (Santa Cruz Biotechnology Inc., catalog sc-393406), and TAP2 (Santa Cruz Biotechnology Inc., catalog sc-515576) were purchased. At the same protein concentration, GAPDH (Cell Signaling Technology, catalog 21185) was used as the control for normalizing during quantification of the blots. In brief, 20 μg of lysate supernatant was separated by 10% sodium dodecyl sulfate-polyacrylamide gel electrophoresis and transferred to a nitrocellulose membrane. The blots were developed using enhanced chemiluminescent reagents (Bio-Rad Laboratories) and the ChemiDoc XRS⁺Molecular Imager (Bio-Rad Laboratories).

### Zebrafish Husbandry and Maintenance

Zebrafish (Danio rerio) were raised in a circulating aquarium system on a 14-hour light/10-hour dark cycle at 28.5° C., following standard husbandry procedures.

Morpholino and mRNA Microinjection

The morpholino antisense oligo (MO) 5′-CGACACTTCCTCTGAGCTACTCATG-3′ (SEQ ID NO: 23) was synthesized to block the translation initiation of csnk2b, and the 5-mismatch oligo 5′-CGAGAGTTCGTCTGACCTAGTCATG-3′ (SEQ ID NO: 24) was synthesized as a specificity control (Gene Tools). For synthesizing CSNK2B rescue mRNA that is resistant to the translation blocking MO, the full-length csnk2b coding sequence with 4 base pairs of silent mutations in the MO recognition region was cloned into the pCS2-vector (Azenta Life Sciences), in which the first 24 base pairs of the CSNK2B coding sequence became 5′-ATGAGTAGCTCAGAAGAGGTCTCC-3′ (SEQ ID NO: 25). The csnk2b capped mRNA was synthesized using the mMESSAGE mMACHINE Kit (Ambion, AM1340). Microinjection was performed on WT AB embryos at the 1- to 2-cell stages. Unless otherwise indicated, each embryo was injected with 5 ng of CSNK2B MO and 50 pg of CSNK2B mRNA for KD and rescue, respectively.

### Hemoglobin Staining

Embryos at 2 dpf were dechorionated and anesthetized with 0.016% tricaine (Fluka, A5040), followed by a 15-minute incubation in 0.6 mg/mL o-dianisidine solution (SigmaAldrich, D9143). This solution was prepared in 0.65% H2 O2 (EMD, HX0647-3), 40% ethanol (KOPTEC, 89125), and 10 mM sodium acetate (Fisher Chemical, S210-500) at room temperature. Stained embryos were washed twice with 1×PBS (Gibco, Thermo Fisher Scientific, 14200166) and then fixed in 4% paraformaldehyde (PFA) (SigmaAldrich, P6148). Hemoglobin signal was observed under a light microscope and quantified according to the area and intensity in the heart and common cardinal vein; embryos were categorized into normal, medium, and low hemoglobin levels.

### Example 1—Differences in Long Noncoding Expression Among CMS and Non-CMS Subjects

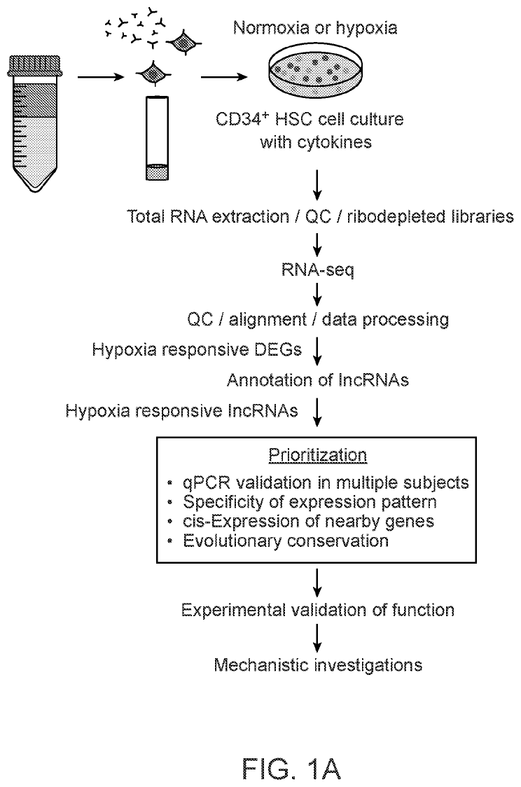

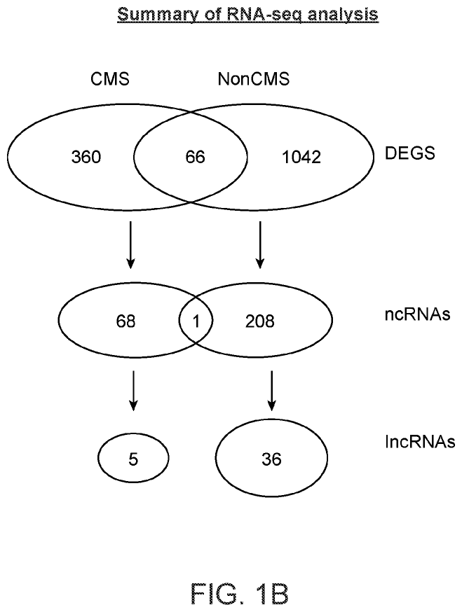

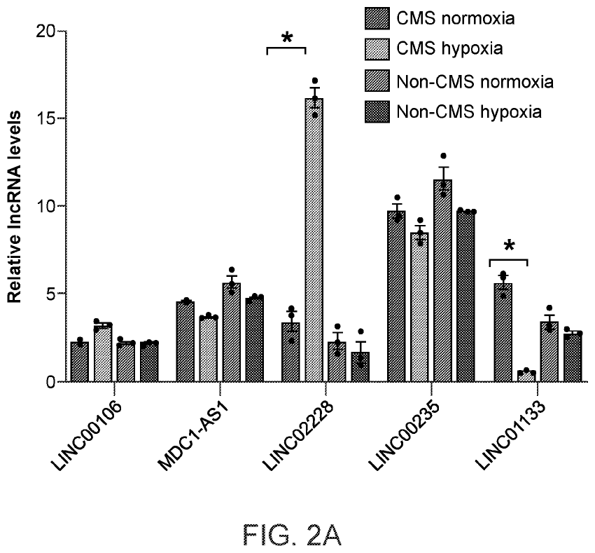

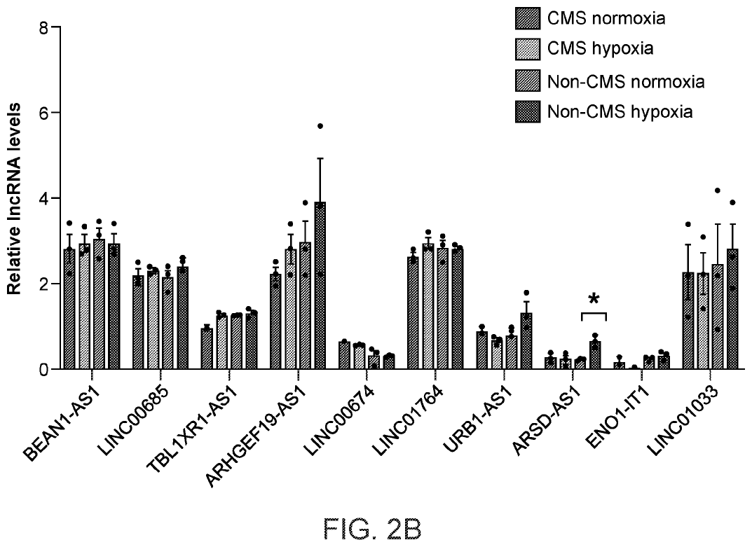

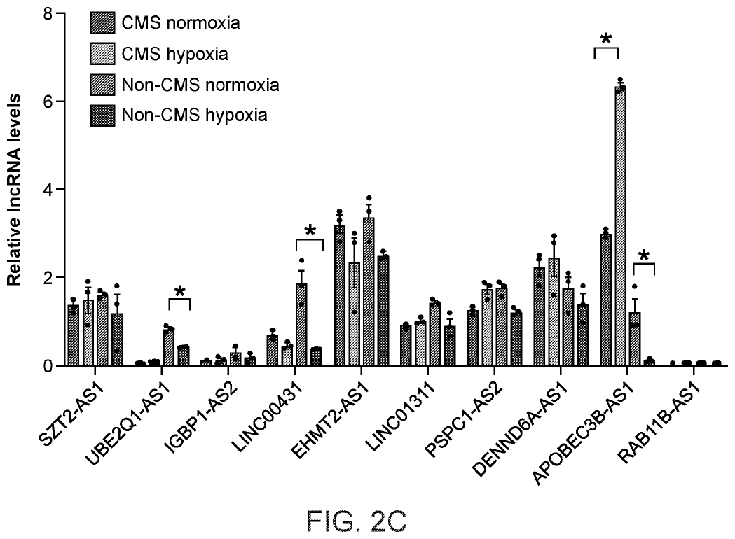

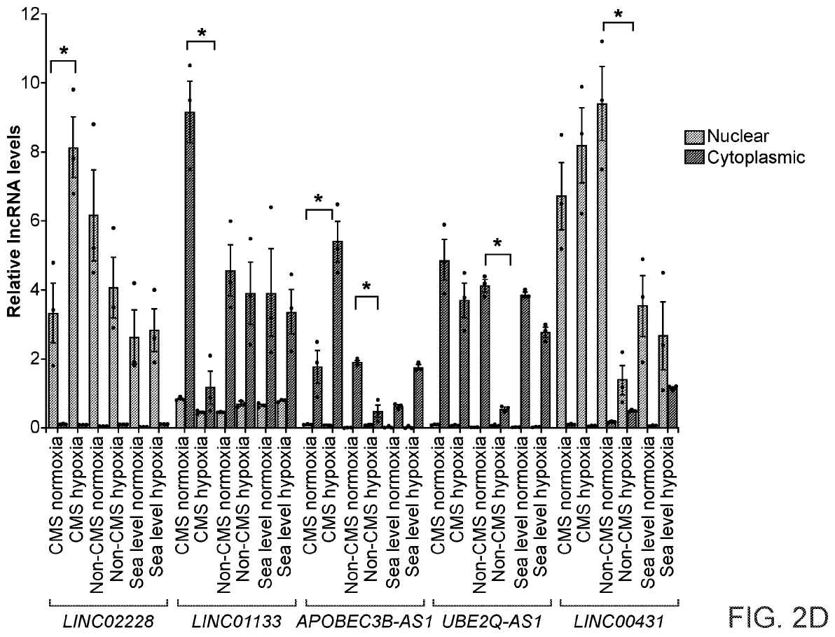

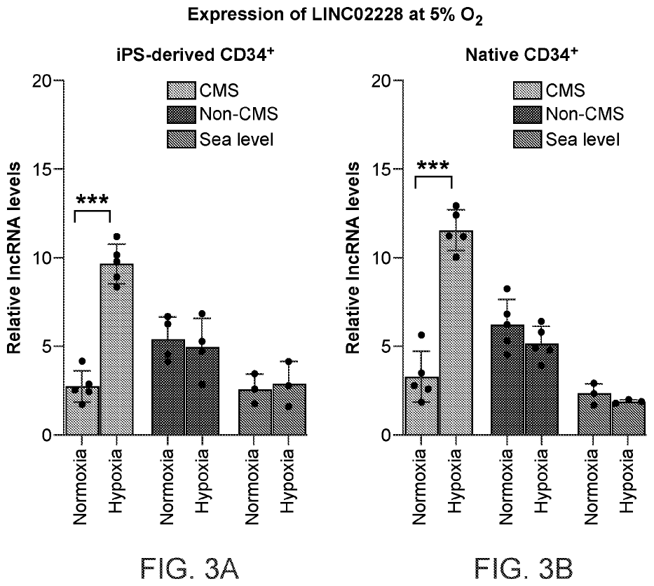

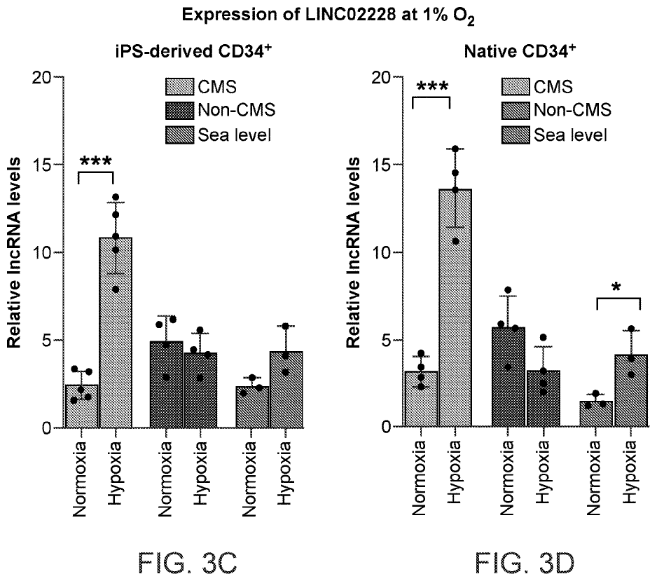

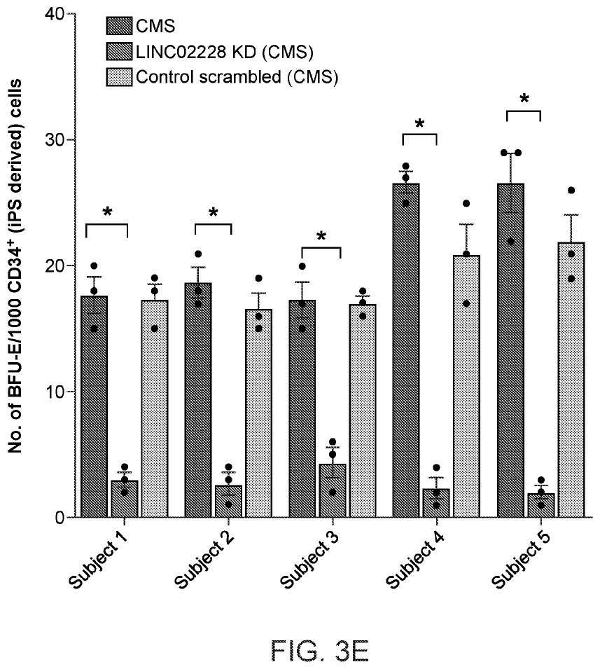

PBMC-derived native CD34⁺ cells that were isolated from CMS (n=4) and non-CMS (n=2) subjects were exposed to either 5% O₂, a hypoxia level that induces significant EE in CMS, or normoxia (as controls) (FIG. 1A). The screening experiment was started with pooling samples for each group and performing an RNA-Seq on the pooled samples to determine the transcriptomic response of the CMS and non-CMS cells to hypoxia. A total of 360 differentially expressed genes (DEGs) in the CMS cells and 1,042 DEGs in the non-CMS cells (>2-fold) were identified, including both coding and lncRNAs. Among these DEGs, 5 differentially expressed lncRNAs in CMS and 36 differentially expressed lncRNAs in non-CMS were identified with no overlap (FIG. 1B and Table 2). Such distinct differences in the transcriptional response in lncRNAs between CMS and non-CMS suggested that specific lncRNA mechanisms are involved in stimulating or inhibiting hypoxia induced erythropoiesis. To verify the expression of a subset (top upregulated and downregulated subsets of lncRNAs based on fold change; Table 2) of the candidate lncRNAs, real-time PCR was performed using iPSC-derived CD34⁺ cells that were generated from CMS (n=3) and non-CMS (n=3) subjects (FIGS. 2A-2C). Significantly altered lncRNAs under hypoxia included HIKER/LINC02228, LINC01133, ARSDAS1, UBE2Q1-AS1, RAB11-B-AS1, LINC00431, and APOBEC3B-AS1 (FIGS. 2A-2D). In addition, the hypoxia-induced upregulation of HIKER/LINC02228 was confirmed in another set of iPSC-derived and PBMC-derived native CD34+ cells obtained from CMS (n=5) or non-CMS (n=5) subjects (FIGS. 3A-3D) at 5% O₂. HIKER/LINC02228 expression levels were further tested in iPSC derived and PBMC native CD34+ levels at 1% O₂ and a similar response was found in all the samples (FIGS. 3A-3D). Since lncRNAs can be predominantly either in the cytosol or in the nucleus, the cellular distribution of the top 5 significantly changed lncRNAs were verified demonstrating that HIKER/LINC02228 and LINC00431 are predominantly located in the nucleus whereas LINC01133, UBE2Q1-AS1, and APOBEC3B-AS1 are mostly cytoplasmic in location (FIG. 2D). Since the current study is focused on transcriptional regulation, nuclear-specific approaches were applied to studying the functional role of the nuclear lncRNAs (i.e., HIKER/LINC02228 and LINC0043T) in regulating erythropoiesis.

TABLE 2
List of candidate lncRNAs altered by hypoxia treatment

| | | | | | |
|---|---|---|---|---|---|
| ID | Symbol | Entrez Gene Name | log2 FC | 1stRPKM | 2ndRPKM |

CMS

| | | | | | |
|---|---|---|---|---|---|
| ENSG00000236871.6 | LINC00106 | long intergenic non-protein coding RNA 106 | 1.964 | 0.662 | 2.893 |
| ENSG00000224328.1 | MDC1-AS1 | MDC1 antisense RNA 1 | 1.223 | 4.427 | 10.443 |
| ENSG00000251273.1 | LINC02228 | long intergenic non-protein coding RNA 2228 | 1.009 | 2.203 | 4.544 |
| ENSG00000277142.1 | LINC00235 | long intergenic non-protein coding RNA 235 | −1.056 | 2.407 | 1.140 |
| ENSG00000224259.5 | LINC01133 | long intergenic non-protein coding RNA 1133 | −1.956 | 3.665 | 0.902 |

nonCMS

| | | | | | |
|---|---|---|---|---|---|
| ENSG00000280851.6 | BEAN1-AS1 | BEAN1 antisense RNA 1 | 4.954 | 0.000 | 2.173 |
| ENSG00000226179.5 | LINC00685 | long intergenic non-protein coding RNA 685 | 4.148 | 0.000 | 2.377 |
| ENSG00000231310.3 | TBL1XR1-AS1 | TBL IXR1 antisense RNA 1 | 2.751 | 0.788 | 5.712 |
| ENSG00000234166.1 | ARHGEF19-AS1 | ARHGEF19 antisense RNA 1 | 2.468 | 0.279 | 2.163 |
| ENSG00000237654.3 | LINC00674 | long intergenic non-protein coding RNA 674 | 2.101 | 0.584 | 2.773 |
| ENSG00000267308.1 | LINC01764 | | 1.919 | 1.008 | 4.056 |
| ENSG00000256073.3 | URB1-AS1 | URB1 antisense RNA 1 (head to head) | 1.571 | 1.327 | 4.081 |
| ENSG00000229851.1 | ARSD-AS1 | ARSD antisense RNA 1 | 1.530 | 1.652 | 4.905 |
| ENSG00000236269.1 | ENO1-IT1 | | 1.488 | 1.153 | 3.373 |
| ENSG00000249069.7 | LINC01033 | | 1.346 | 1.264 | 3.281 |
| ENSG00000244265.1 | SIAH2-AS1 | SIAH2 antisense RNA 1 | 1.248 | 3.752 | 9.075 |
| ENSG00000268692.3 | RAET1E-AS1 | RAET 1E antisense RNA 1 | 1.146 | 0.912 | 2.074 |
| ENSG00000264261.8 | PSMB6-AS1 | PSMB8 antisense RNA 1 (head to head) | 1.135 | 3.864 | 8.518 |
| ENSG00000246067.7 | RAB30-AS1 | RAB30 antisense RNA 1 (head to head) | 1.130 | 1.085 | 2.402 |
| ENSG00000225378.5 | TMEM246-AS1 | TMEM246 antisense RNA 1 | 1.103 | 1.140 | 2.524 |
| ENSG00000242797.3 | GLYCTK-AS1 | GLYCTK antisense RNA 1 | 1.085 | 1.013 | 2.194 |
| ENSG00000245148.2 | ARAP1-AS2 | ARAP1 antisense RNA 2 | 1.026 | 1.848 | 3.793 |
| ENSG00000233661.1 | SPIN4-AS1 | SPIN4 antisense RNA 1 | −1.087 | 7.193 | 3.344 |
| ENSG00000279873.2 | LINC01126 | long intergenic non-protein coding RNA 1126 | −1.199 | 2.234 | 0.948 |
| ENSG00000264575.1 | LINC00526 | long intergenic non-protein coding RNA 526 | −1.268 | 2.353 | 0.941 |
| ENSG00000272414.5 | FAM47E-STBD1 | FAM47E-STBO1 readthrough | −1.383 | 6.766 | 2.197 |
| ENSG00000248866.1 | USP46-AS1 | USP46 antisense RNA 1 | −1.410 | 2.865 | 1.035 |
| ENSG00000223534.1 | HLA-DOB1-AS1 | MLA-DQB1 antisense RNA 1 | −1.438 | 4.552 | 1.598 |
| ENSG00000224101.1 | ELMO1-AS1 | ELMO1 antisense RNA 1 | −1.438 | 8.848 | 2.441 |
| ENSG00000237883.1 | DGUOK-AS1 | DGUOK antisense RNA 1 | −1.438 | 2.692 | 0.941 |
| ENSG00000269521.1 | INO80-AS1 | | −1.458 | 4.132 | 1.429 |
| ENSG00000229372.1 | SZT2-AS1 | SZT2 antisense RNA 1 | −1.523 | 2.981 | 0.970 |
| ENSG00000229780.1 | UBE2O1-AS1 | UBE2Q1 antisense RNA 1 | −1.760 | 16.882 | 4.868 |
| ENSG00000220925.2 | IGBP1-AS2 | | −1.776 | 8.462 | 2.380 |
| ENSG00000225760.1 | LINC00431 | long intergenic non-protein coding RNA 431 | −1.924 | 3.673 | 0.893 |
| ENSG00000237080.2 | EHMT2-AS1 | | −1.932 | 6.079 | 1.455 |
| ENSG00000260924.2 | LINC01311 | long intergenic non-protein coding RNA 1311 | −2.491 | 2.333 | 0.378 |
| ENSG00000228352.2 | PSPC1-AS2 | | −2.608 | 4.238 | 0.541 |
| ENSG00000239801.1 | DENND6A-AS1 | | −2.626 | 6.735 | 0.932 |
| ENSG00000249310.2 | APOBEC3B-AS1 | APOBEC3B antisense RNA 1 | −3.497 | 2.129 | 0.098 |
| ENSG00000269386.5 | RAB11B-AS1 | RAB11B antisense RNA 1 | −4.345 | 2.333 | 0.059 |

### Example 2—HIKER/LINC02228 Regulates Erythropoiesis in CMS Subjects

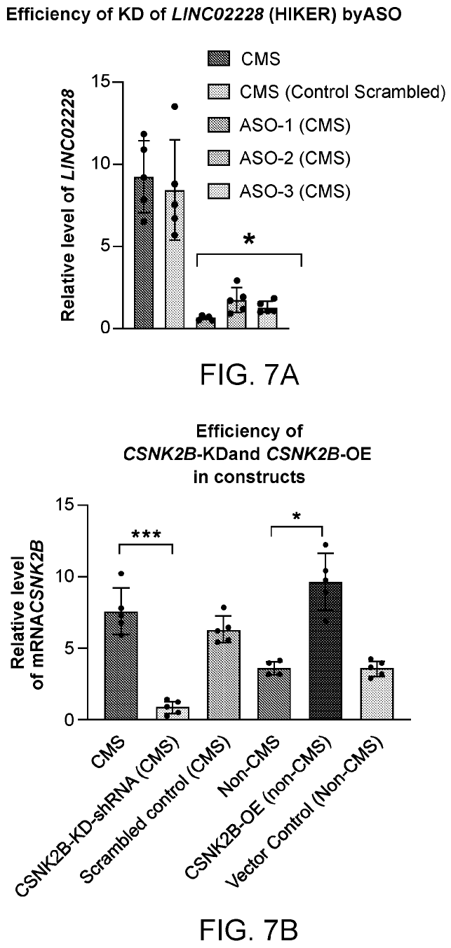

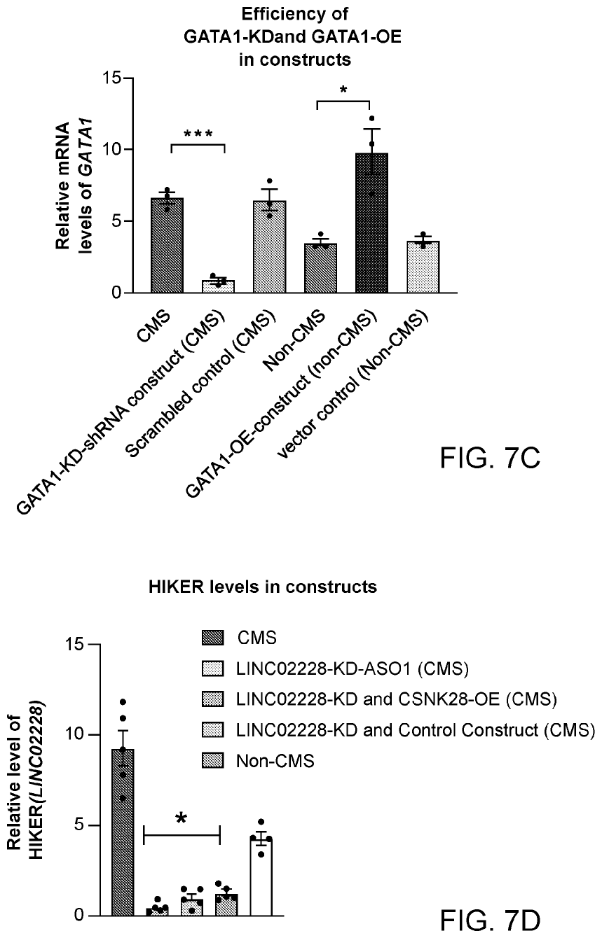

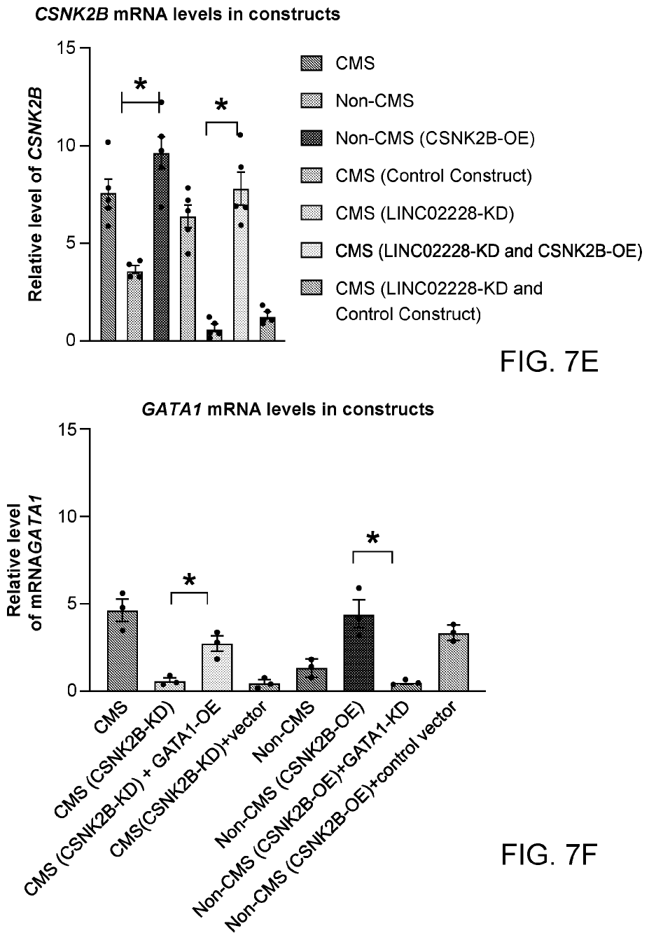

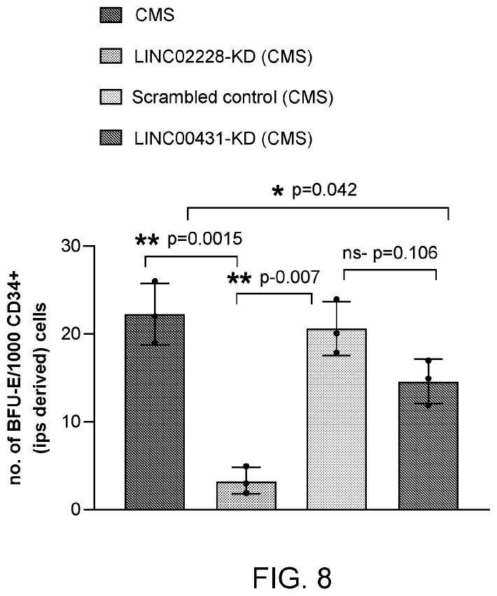

In order to assess the functional role of the candidate nuclear lncRNAs, lncRNAs that were upregulated in CMS cells (i.e., HIKER/LINC02228) (FIG. 2A, FIGS. 3A-3D, and FIG. 8) and downregulated in non-CMS cells (i.e., LINC00431) (FIG. 8) were selected. By testing the function (by colony-forming assay) of these 2 lncRNAs in CMS cells, the functionality as well as specificity of each candidate lncRNA was ascertained. Using the efficient available KD strategy for nuclear lncRNAs, the 2 lncRNAs were downregulated using antisense oligonucleotide (ASO). The downregulation (>80%, FIGS. 7A-7F) of HIKER/LINC02228 in the CMS cells led to a significant reduction of burst-forming unit-erythroid (BFU-E) colonies (P<0.0001) (FIG. 3E), but only a modest suppression (P=0.04) (FIG. 8) by LINC00431 with no statistical significance against the scrambled control (P>0.05) (FIG. 8). These results demonstrate a critical role of HIKER/LINC02228 in regulating erythroid progenitors (BFU-E) in CMS cells under hypoxia. The strong inhibition of BFU-E progenitors with KD of HIKER/LINC02228, but not with KD of LINC00431, also strongly suggests a specific role of HIKER/LINC02228 in regulating EE in CMS subjects.

### Example 3—CSNK2B is a Critical Mediator of HIKER/LINC02228 for Driving Erythropoiesis Under Hypoxia

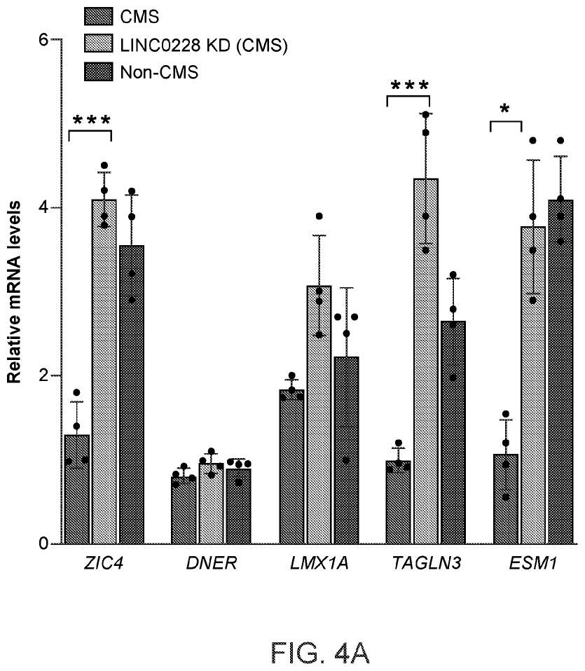

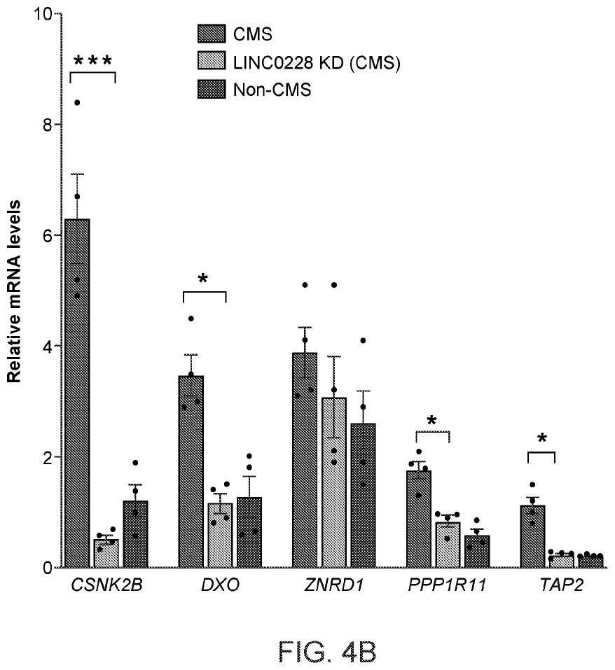

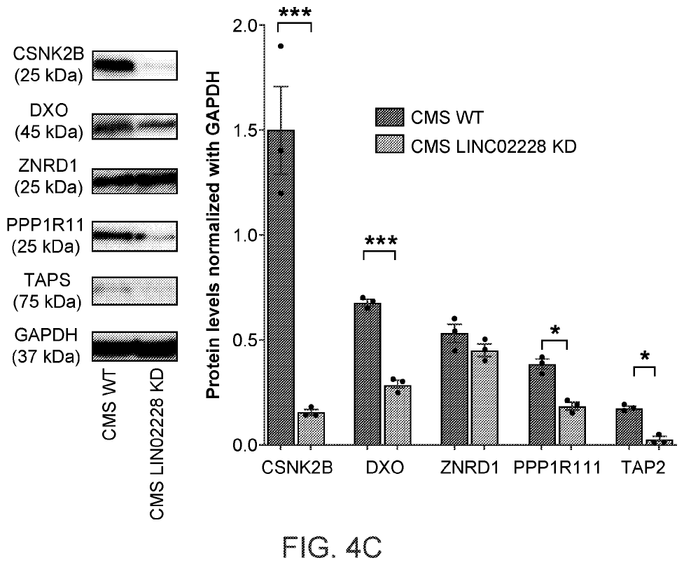

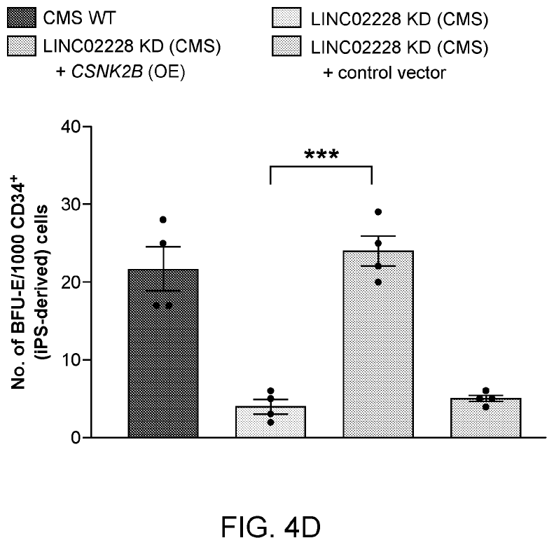

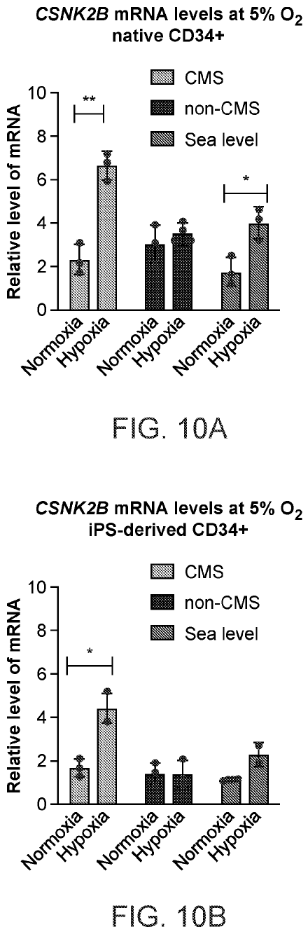

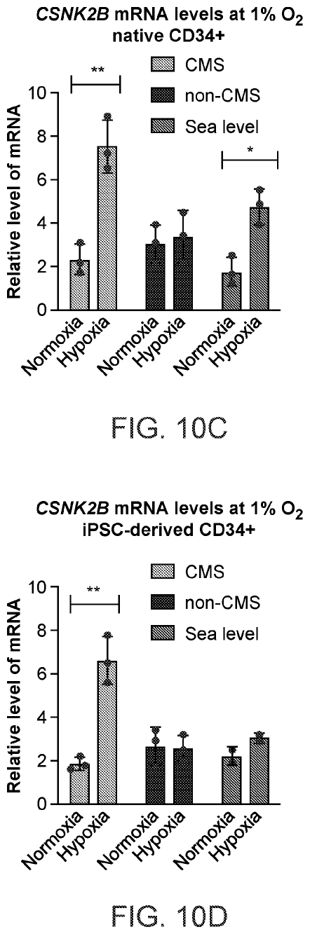

To determine potential downstream factors mediating the function of HIKER/LINC02228 in erythropoiesis. DEGs in the CMS cells following the KD of HIKER/LINC02228 or LINC00431 were next identified. Compared with controls, a total of 363 DEGs with HIKER/LIN02228 KD and a total of 361 DEGs with LIN00431 KD were identified. Since HIKER/LINC02228 KD specifically decreased hypoxia-induced BFU-E colonies, but LINC00431 KD had no significant effect (FIGS. 3A-3E and FIG. 8), the list of LINC00431 KD DEGs was used as an additional filtering strategy for identifying the DEG candidates that were specifically altered by HIKER/LINC02228 KD, as these would be more likely to be functional mediators of LINC02228 in excessive erythropoiesis. To do so, the list of DEGs following HIKER/LINC02228 KD or LINC00431 KD was compared and 238 DEGs that were common in both lists were removed. This filtering process generated a list of 125 candidate DEGs that were specifically altered by HIKER/LINC02228 KD (Table 3), which was focused on in the follow-up studies. The top upregulated candidates (e.g, ZIC4, DNER, LMX1A TAGLN3, and ESM1) were then verified using quantitative PCR (qPCR), and the most downregulated candidates (e.g., CSNK2B, DXO, ZNRD1. PPP1R11 and TAP2) were verified with both qPCR and Western blot analysis (FIGS. 4A-4C, FIGS. 10A-10D, and Table 4).

Through filtering and experimental validation processes, CSNK2B was confirmed to be a promising candidate with the most significant (P<0.01) alterations by both qPCR and Western blotting. In order to functionally assess (through colony-forming assay) whether CSNK2B is a critical mediator of HIKER/LINC02228, a rescue experiment (FIG. 4D) was performed, in which HIKER/LINC02228 in CMS cells was knocked down, and on such a background, CSNK2B was overexpressed (OE). Indeed, CSNK2B OE completely rescued the effect of HIKER/LINC02228 KD, demonstrating that CSNK2B is a critical downstream effector mediating the function of HIKER/LINC02228 (FIG. 4D).

### Example 4—CSNK2B is an Erythropoietic Regulator in CMS and Non-CMS Cells

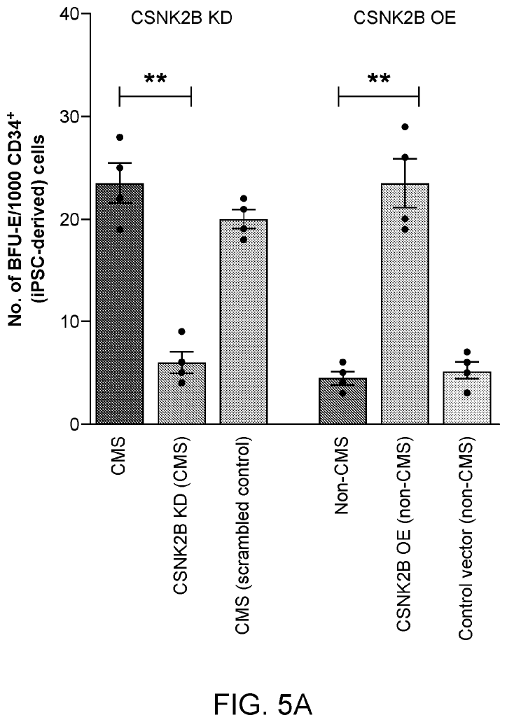

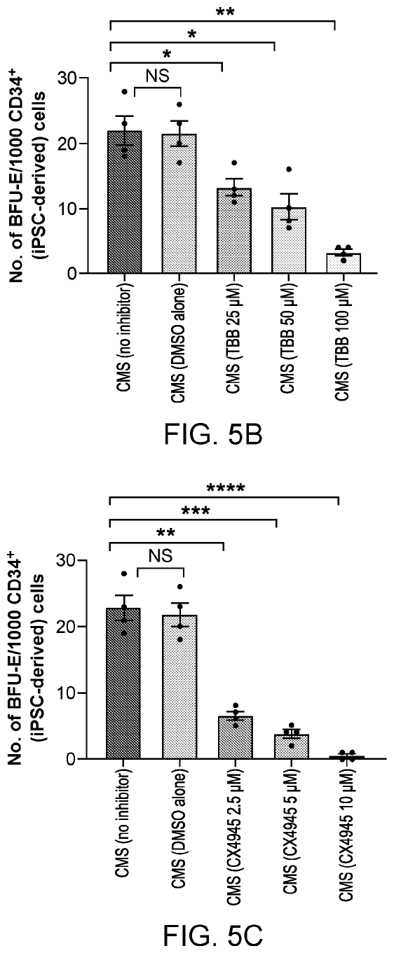

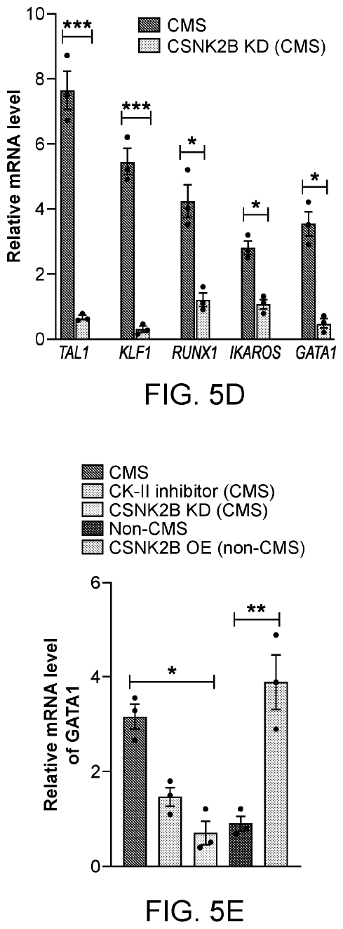

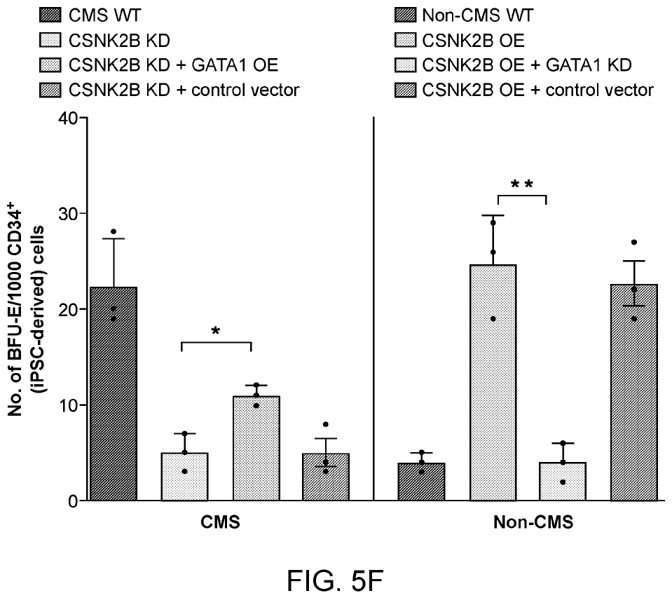

The role of CSNK2B in erythropoiesis was further evaluated using in vitro erythroid platform. On the one hand, when CSNK2B expression was downregulated in CMS cells, there was a remarkable decrease in erythropoiesis in response to hypoxia. On the other hand, CSNK2B OE in the non-CMS cells resulted in an excessive erythropoietic response to hypoxia, which phenocopied the CMS cells (FIG. 5A). In order to test the hypothesis that the role of CSNK2B in EE is achieved through its regulation of CK2 activity, specific inhibitors (TBB and CX-4945) of the CK2 were used and their effect on erythroid colony production was studied. Consistently with the KD results, significant changes were observed in the colony numbers in a dose-dependent manner with the inhibitors (FIGS. 5B and 5C) (P<0.0001, multiple comparisons by Tukey's test between control and inhibitor at various dosages). Collectively, RNAi and inhibitor results confirm an important role of CK2 in regulating the erythropoietic response of CMS and non-CMS cells under hypoxia.

TABLE 3
List of specific LINC02228 downstream target transcripts

| Gene_Symbol | baseMean | log2FoldChange | IfcSE | stat | pvalue |
|---|---|---|---|---|---|
| ZIC4 | 99.632 | 5.571 | 2.422 | 2.300 | 0.021 |
| DNER | 116.940 | 4.348 | 2.038 | 2.137 | 0.033 |
| LMX1A | 87.065 | 4.337 | 2.117 | 2.048 | 0.041 |
| CDH18 | 79.390 | 4.197 | 2.107 | 1.992 | 0.046 |
| TAGLN3 | 84.095 | 4.050 | 2.045 | 1.980 | 0.048 |
| ESM1 | 141.557 | 3.899 | 1.865 | 2.091 | 0.037 |
| SLC16A14 | 117.895 | 3.875 | 1.902 | 2.038 | 0.042 |
| MMRN1 | 467.196 | 3.860 | 1.663 | 2.321 | 0.020 |
| GPM6A | 137.902 | 3.859 | 1.880 | 2.075 | 0.038 |
| CALB1 | 116.387 | 3.720 | 1.861 | 1.999 | 0.046 |
| CNTN2 | 147.995 | 3.646 | 1.785 | 2.043 | 0.041 |
| MAP2 | 497.426 | 3.640 | 1.599 | 2.276 | 0.023 |
| MAP6 | 179.603 | 3.600 | 1.732 | 2.078 | 0.038 |
| ILDR2 | 231.583 | 3.587 | 1.684 | 2.130 | 0.033 |
| SLC1A2 | 184.213 | 3.421 | 1.677 | 2.040 | 0.041 |
| FOXJ1 | 280.406 | 3.333 | 1.584 | 2.104 | 0.035 |
| DCX | 265.834 | 3.292 | 1.580 | 2.084 | 0.037 |
| NAV3 | 238.152 | 3.208 | 1.572 | 2.040 | 0.041 |
| NEFL | 593.094 | 3.164 | 1.454 | 2.177 | 0.029 |
| CTNNA2 | 282.317 | 3.104 | 1.517 | 2.046 | 0.041 |
| NELL2 | 500.631 | 2.890 | 1.390 | 2.080 | 0.038 |
| FAM84A | 408.271 | 2.755 | 1.369 | 2.012 | 0.044 |
| GPR98 | 1118.022 | 2.429 | 1.195 | 2.032 | 0.042 |
| KRT7 | 2449.952 | 2.395 | 1.159 | 2.066 | 0.039 |
| IGFBPL1 | 909.744 | 2.304 | 1.168 | 1.973 | 0.049 |
| KIF1A | 1030.216 | 2.274 | 1.152 | 1.975 | 0.048 |
| PAPPA2 | 1998.311 | 2.118 | 1.077 | 1.967 | 0.049 |
| EEF1A1 | 166227.896 | −2.000 | 1.010 | −1.981 | 0.048 |
| EPHA7 | 4509.372 | −2.053 | 1.040 | −1.974 | 0.048 |
| HMGB1 | 5532.229 | −2.206 | 1.087 | −2.030 | 0.042 |
| HERC2P2 | 1705.768 | −2.293 | 1.137 | −2.017 | 0.044 |
| CXCL14 | 1046.689 | −2.294 | 1.157 | −1.983 | 0.047 |
| RSPO2 | 1699.377 | −2.309 | 1.142 | −2.022 | 0.043 |
| GOLGABA | 970.402 | −2.388 | 1.190 | −2.006 | 0.045 |
| FIBIN | 1003.742 | −2.418 | 1.197 | −2.020 | 0.043 |
| EGFLAM | 2963.314 | −2.450 | 1.172 | −2.091 | 0.037 |
| RPL39 | 7599.851 | −2.470 | 1.165 | −2.120 | 0.034 |
| PKD1P1 | 1662.080 | −2.587 | 1.228 | −2.106 | 0.035 |
| DCN | 4747.156 | −2.608 | 1.212 | −2.153 | 0.031 |
| RPS3A | 17051.926 | −2.617 | 1.205 | −2.172 | 0.030 |
| FAM45B | 1154.329 | −2.639 | 1.258 | −2.097 | 0.036 |
| RNU12 | 3100.256 | −2.641 | 1.228 | −2.150 | 0.032 |
| HGF | 678.513 | −2.718 | 1.313 | −2.070 | 0.038 |
| FABP5 | 634.996 | −2.734 | 1.323 | −2.067 | 0.039 |
| PDZRN4 | 541.804 | −2.757 | 1.342 | −2.055 | 0.040 |
| SNORD116-17 | 705.144 | −2.795 | 1.333 | −2.097 | 0.036 |
| SNORD116-19 | 705.144 | −2.795 | 1.333 | −2.097 | 0.036 |
| GATSL2 | 977.185 | −2.799 | 1.314 | −2.130 | 0.033 |
| PI4KAP2 | 646.437 | −2.823 | 1.342 | −2.104 | 0.035 |
| NOMO1 | 3561.968 | −2.832 | 1.281 | −2.210 | 0.027 |
| SERPINA1 | 546.770 | −2.834 | 1.364 | −2.078 | 0.038 |
| SDHAP1 | 470.868 | −2.860 | 1.385 | −2.065 | 0.039 |
| ALOX15 | 927.548 | −2.872 | 1.338 | −2.146 | 0.032 |
| TYRP1 | 951.996 | −2.878 | 1.338 | −2.150 | 0.032 |
| LOC440434 | 501.107 | −2.900 | 1.391 | −2.085 | 0.037 |
| PROK1 | 411.111 | −2.924 | 1.417 | −2.063 | 0.039 |
| BCRP3 | 216.022 | −2.968 | 1.514 | −1.961 | 0.050 |
| PI4KAP1 | 439.110 | −3.056 | 1.449 | −2.109 | 0.035 |
| ZNF286B | 618.774 | −3.099 | 1.430 | −2.167 | 0.030 |
| HLA-B | 777.458 | −3.116 | 1.418 | −2.197 | 0.028 |
| ODAM | 283.023 | −3.118 | 1.518 | −2.054 | 0.040 |
| COL21A1 | 845.460 | −3.165 | 1.427 | −2.219 | 0.027 |
| HLA-DPB1 | 490.874 | −3.174 | 1.471 | −2.157 | 0.031 |
| PDIA3P1 | 876.702 | −3.189 | 1.431 | −2.228 | 0.026 |
| LOC100288162 | 607.773 | −3.232 | 1.469 | −2.200 | 0.028 |
| MIR6723 | 388.199 | −3.235 | 1.512 | −2.139 | 0.032 |
| ARHGEF5 | 570.506 | −3.277 | 1.486 | −2.205 | 0.027 |
| GCSH | 763.804 | −3.305 | 1.472 | −2.246 | 0.025 |
| LOC440300 | 557.227 | −3.376 | 1.515 | −2.228 | 0.026 |
| LOC100288778 | 474.025 | −3.566 | 1.581 | −2.255 | 0.024 |
| APOC1P1 | 106.132 | −3.836 | 1.902 | −2.017 | 0.044 |
| PLEKHM1 | 404.598 | −3.846 | 1.671 | −2.301 | 0.021 |
| NPIPA1 | 348.955 | −3.848 | 1.689 | −2.278 | 0.023 |
| ANXA2P1 | 86.201 | −3.880 | 1.968 | −1.972 | 0.049 |
| ARHGEF34P | 354.652 | −4.002 | 1.726 | −2.318 | 0.020 |
| LOC643387 | 123.552 | −4.152 | 1.950 | −2.129 | 0.033 |
| AHSG | 287.192 | −4.259 | 1.819 | −2.342 | 0.019 |
| FAM72A | 156.115 | −4.274 | 1.930 | −2.214 | 0.027 |
| RPL23P8 | 352.000 | −4.317 | 1.805 | −2.392 | 0.017 |
| ROCK1P1 | 185.258 | −4.331 | 1.910 | −2.267 | 0.023 |
| MTRNR2L2 | 138.920 | −4.417 | 1.992 | −2.218 | 0.027 |
| ATP8B5P | 73.747 | −4.421 | 2.162 | −2.045 | 0.041 |
| GTF2IRD2 | 267.167 | −4.497 | 1.888 | −2.382 | 0.017 |
| NACAP1 | 65.492 | −4.621 | 2.255 | −2.049 | 0.040 |
| ARHGAP27 | 157.708 | −4.699 | 2.034 | −2.310 | 0.021 |
| LOC642236 | 121.359 | −4.737 | 2.105 | −2.251 | 0.024 |
| CRSP8P | 45.242 | −4.904 | 2.476 | −1.980 | 0.048 |
| PMS2L2 | 56.552 | −4.904 | 2.386 | −2.056 | 0.040 |
| HLA-DPA1 | 242.897 | −5.252 | 2.079 | −2.526 | 0.012 |
| GOLGA8N | 44.876 | −5.319 | 2.601 | −2.045 | 0.041 |
| ANKRD20A4 | 35.618 | −5.577 | 2.797 | −1.994 | 0.046 |
| FAM35DP | 215.075 | −5.586 | 2.177 | −2.566 | 0.010 |
| RAB6C | 184.930 | −5.632 | 2.219 | −2.539 | 0.011 |
| GOLGA6L6 | 55.821 | −5.641 | 2.598 | −2.171 | 0.030 |
| NPIP85 | 170.199 | −5.665 | 2.245 | −2.523 | 0.012 |
| CXADRP2 | 47.247 | −5.992 | 2.781 | −2.154 | 0.031 |
| ZNF812 | 28.412 | −6.262 | 3.183 | −1.967 | 0.049 |
| LOC100132057 | 29.096 | −6.297 | 3.181 | −1.980 | 0.048 |
| HLA-DOA | 175.259 | −6.302 | 2.384 | −2.644 | 0.008 |
| CTAGE4 | 141.010 | −6.577 | 2.509 | −2.622 | 0.009 |
| ZNRD1-AS1 | 151.271 | −6.679 | 2.512 | −2.659 | 0.008 |
| LOC613037 | 129.699 | −6.874 | 2.609 | −2.634 | 0.008 |
| POM121L1P | 43.461 | −6.882 | 3.139 | −2.192 | 0.028 |
| PRR21 | 50.986 | −7.114 | 3.126 | −2.275 | 0.023 |
| DDX11L10 | 51.670 | −7.133 | 3.125 | −2.282 | 0.022 |
| CFC1B | 115.653 | −7.297 | 2.766 | −2.638 | 0.008 |
| DDX11L9 | 58.510 | −7.314 | 3.117 | −2.346 | 0.019 |
| LINC01001 | 117.705 | −7.322 | 2.767 | −2.647 | 0.008 |
| TSPY3 | 64.667 | −7.459 | 3.111 | −2.397 | 0.017 |
| TNXA | 65.351 | −7.474 | 3.111 | −2.403 | 0.016 |
| CST4 | 66.719 | −7.504 | 3.109 | −2.413 | 0.016 |
| TSPY4 | 86.557 | −7.882 | 3.097 | −2.545 | 0.011 |
| LOC100190986 | 89.293 | −7.927 | 3.096 | −2.560 | 0.010 |
| MICB | 102.974 | −8.133 | 3.092 | −2.630 | 0.009 |
| FAM157B | 22.574 | −8.391 | 4.231 | −1.983 | 0.047 |
| RNU1-13P | 25.310 | −8.556 | 4.188 | −2.043 | 0.041 |
| REREP3 | 32.151 | −8.901 | 4.107 | −2.167 | 0.030 |
| MOG | 43.780 | −9.347 | 4.021 | −2.325 | 0.020 |
| PRRT1 | 43.780 | −9.347 | 4.021 | −2.325 | 0.020 |
| TCEB3CL | 43.780 | −9.347 | 4.021 | −2.325 | 0.020 |
| TAP2 | 486.731 | −10.378 | 5.221 | −1.988 | 0.047 |
| PPP1R11 | 1756.707 | −11.230 | 5.259 | −2.135 | 0.033 |
| ZNRD1 | 1242.615 | −11.731 | 5.518 | −2.126 | 0.034 |
| DXO | 673.797 | −13.290 | 6.767 | −1.964 | 0.050 |
| CSNK2B | 6345.321 | −16.526 | 6.766 | −2.442 | 0.015 |

TABLE 4
Top 5 candidate genes comparing KD-
HIKER/LINC02228 versus controls

| Gene | log₂ fold change | P value |
|---|---|---|
| ZIC4 | 5.5713 | 0.0214 |
| DNER | 4.3483 | 0.0326 |
| LMX1A | 4.3372 | 0.0405 |
| TAGLN3 | 4.0498 | 0.0477 |
| ESM1 | 3.8990 | 0.0366 |
| TAP2 | −10.3778 | 0.0468 |
| PPP1R11 | −11.2299 | 0.0327 |
| ZNRD1 | −11.7306 | 0.0335 |
| DXO | −13.2904 | 0.0495 |
| CSNK2B | −16.5255 | 0.0146 |

Top up- (roman) and downregulated (bold) genes based on fold change and P values.

### Example 5—CSNK2B Mediates the High-Altitude Erythropoietic Response in Part Through GATA1

In order to determine how CSNK2B regulates erythropoiesis, RNA-Seq of CNSK2B KD (CMS) versus control (CMS, no KD) was performed. Remarkably, several critical TFs (e.g., TAL1, KLF1, and GATA1) as well as the erythropoietin receptor (EPOR, a target of HIF1A) were found to be downregulated (>2-fold) by CSNK2B KD in CMS cells (FIG. 5D). Since (a) GATA1 is a major erythroid-specific TF and can regulate the expression of other erythroid target genes such as TAL1 and KLF1 and (b) it has been shown that GATA1 plays a critical role in regulating erythropoiesis in CMS cells, experiments were further performed to investigate whether CSNK2B functions via GATA1. First, the expression levels of GATA1 in CMS cells and non-CMS cells after CSNK2B KD were measured. In CMS cells, the KD of CSNK2B as well as pharmacologic inhibition of CK2 resulted in downregulation (about 2-fold, P<0.05) of GATA1 levels (FIG. 5E). In the non-CMS cells, however, OE of CSNK2B led to upregulation (about 3-fold, P<0.001) of GATA1 levels (FIG. 5E). Second, in order to show that there is a functional interaction between CSNK2B and GATA1, double mutants of CSNK2B and GATA1 were used to analyze their effect on colony formation. GATA1OE was observed to be able to partially rescue the erythropoietic suppression caused by CSNK2B in CMS cells (FIG. 5F). In non-CMS cells, the KD of GATA1 led to a large (>5-fold) decrease in the excessive erythropoietic response (BFU-E colonies) caused by CSNK2B OE in these cells (FIG. 5F). The control vectors by themselves did not affect the phenotypes, implying an important role of GATA1 in this experiment (FIG. 5F). These results confirm a partial role of GATA1 as a downstream mediator of CSNK2B in regulating erythropoiesis in CMS and non-CMS cells under hypoxia.

### Example 6—CSNK2B KD Induces Severe Hemoglobinization Defect in Zebrafish Embryos

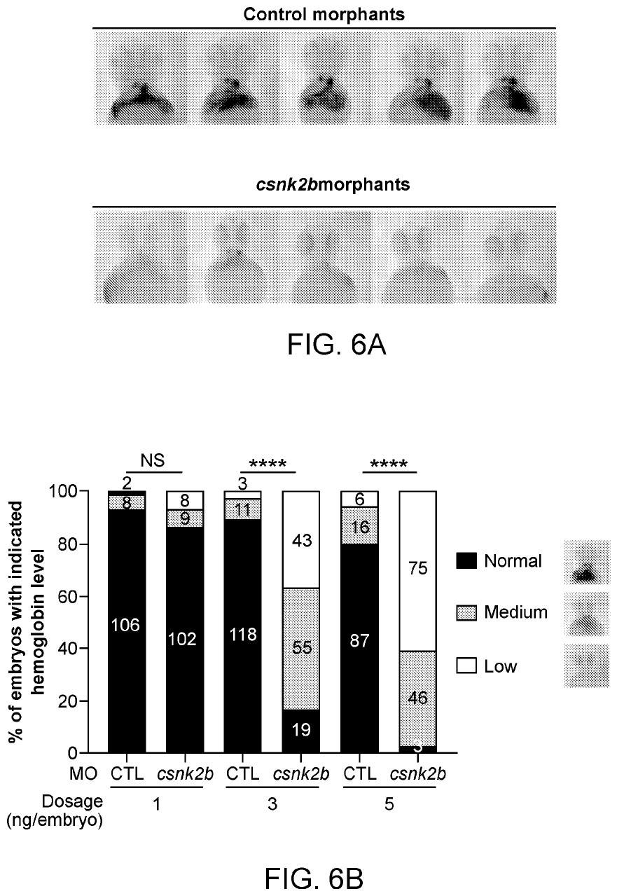

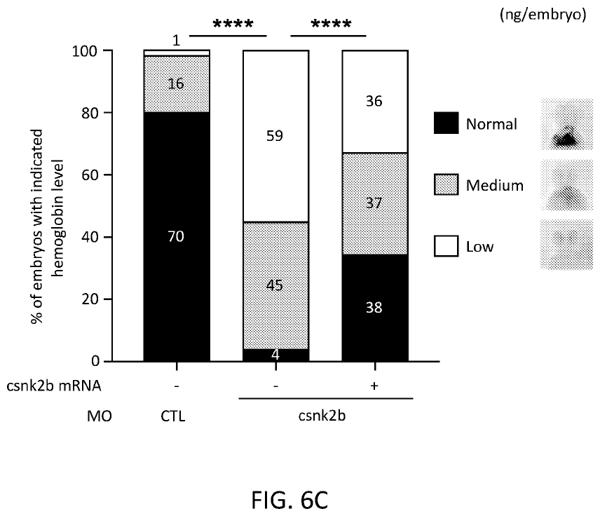

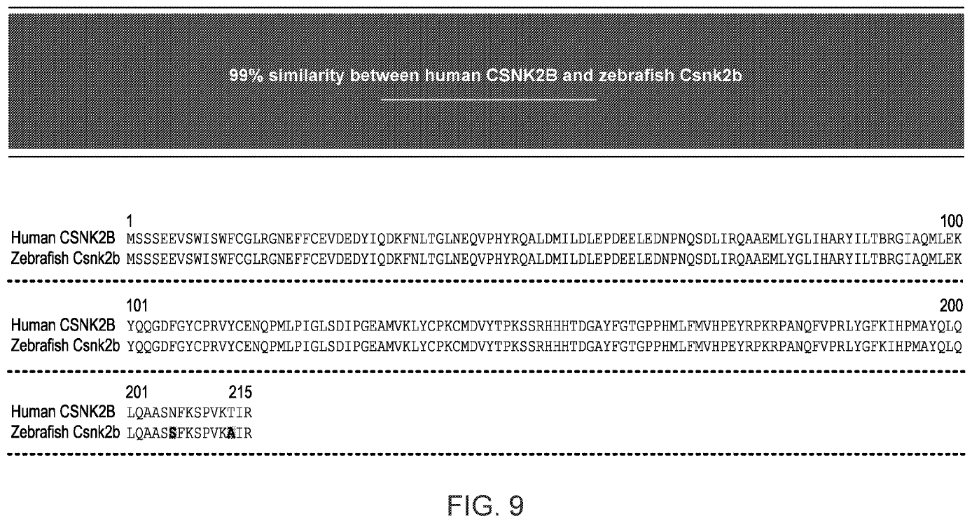

Since the CSNK2B protein sequence is 99% conserved between humans and zebrafish (FIG. 9), the role of CSNK2B in erythropoiesis was assessed in vivo during zebrafish development, the expression of csnk2b in zebrafish embryos was knocked down using a morpholino antisense oligonucleotide (MO) that blocks the translation of csnk2b. Compared with controls, csnk2b-KD embryos displayed a remarkable decrease in hemoglobin at 3 ng and 5 ng MO dosage, 2 days post fertilization (dpf) (FIGS. 6A-6B). A few of the morphants displayed normal iron incorporation at these dosages (FIGS. 6A-6B). On the other hand, more than 97% of morphants showed moderate or low iron staining, indicating a key role of CSNK2B in the maturation of the RBC lineage. Moreover, a dose-dependent increase in phenotype severity was found (FIG. 6B), with a minimal impact on hemoglobin levels at 1 ng dose, but increasing severity in hemoglobin levels at 3 ng and 5 ng. Furthermore, hemoglobin levels in csnk2b morphants were rescued via co-injection of csnk2b mRNA (FIG. 6C). Taken together, these findings indicate that CSNK2B plays a key role in erythropoiesis.

The results show distinct expressional changes in lncRNAs under hypoxia in CMS and non-CMS cells. It is also proved, for what is believed the first time, that the lncRNA HIKER/LINC02228 regulates the excessive erythropoiesis of Monge's disease (FIGS. 3A-3E) and that its action is mediated through CSNK2B, a casein kinase. Furthermore, in vivo KD of CSNK2B in zebrafish results in severe reduction in hemoglobin, further proving its vital role in erythropoiesis.

### Example 7—DEGs with CSNK2B-KD

For LINC00228 and its downstream target CSNK2B, RNA-seq analysis was performed to identify differential expressed genes (DEGs) through comparing CSNK2B-KD versus Controls. These set of DEGs are candidate downstream mediators regulating the function of CSNK2B in erythropoiesis. Among the identified genes exhibiting at least a 2-fold change with statistical significance, 4813 genes were upregulated, and 3310 genes were downregulated. The table below displays the top 100 upregulated or downregulated gene candidates respectively as representative examples.

TABLE 5
Top 100 upregulated genes in response to CSNK2B-KD in CMS cells

| | Control/ | CSNK2B- | Control/ | CSNK2B/ | | E- |
|---|---|---|---|---|---|---|
| Gene_Symbol | Count | KD/Count | Value | Value | Fold_Change | FDR |
| PINCR | 0 | 112 | 0 | 0.199864378 | >10000 up | 1 |
| CSF3 | 0 | 91 | 0 | 0.162389807 | >10000 up | 1 |
| MOBP | 0 | 89 | 0 | 0.1588208 | >10000 up | 1 |
| C9orf64 | 0 | 88 | 0 | 0.157036297 | >10000 up | 1 |
| PNLIPRP3 | 0 | 86 | 0 | 0.15346729 | >10000 up | 1 |
| LOC730100 | 0 | 83 | 0 | 0.14811378 | >10000 up | 1 |
| POTEF | 1 | 364 | 0.00123259 | 0.649559228 | 526.9874014 | 1 |
| DNAH17 | 2 | 669 | 0.002465179 | 1.193832756 | 484.2782576 | 1 |
| CST1 | 1 | 166 | 0.00123259 | 0.29622756 | 240.3294193 | 1 |
| LTA | 1 | 160 | 0.00123259 | 0.28552054 | 231.6428138 | 1 |
| ZSCAN4 | 1 | 134 | 0.00123259 | 0.239123452 | 194.0008566 | 1 |
| SPDYA | 3 | 388 | 0.003697769 | 0.692387309 | 187.2446078 | 1 |
| FNDC7 | 1 | 121 | 0.00123259 | 0.215924908 | 175.1798779 | 1 |
| LOC100506358 | 1 | 115 | 0.00123259 | 0.205217888 | 166.4932724 | 1 |
| ABCA12 | 3 | 344 | 0.003697769 | 0.61386916 | 166.0106832 | 1 |
| ANKRD19P | 1 | 112 | 0.00123259 | 0.199864378 | 162.1499697 | 1 |
| UCN2 | 2 | 218 | 0.002465179 | 0.389021735 | 157.8066669 | 1 |
| MUC16 | 1 | 105 | 0.00123259 | 0.187372854 | 152.0155966 | 1 |
| EOMES | 2 | 180 | 0.002465179 | 0.321210607 | 130.2990828 | 1 |
| MIR205HG | 1 | 89 | 0.00123259 | 0.1588208 | 128.8513152 | 1 |
| LINC01226 | 1 | 88 | 0.00123259 | 0.157036297 | 127.4035476 | 1 |
| LINC00473 | 1 | 76 | 0.00123259 | 0.135622256 | 110.0303366 | 1 |
| LCN15 | 1 | 75 | 0.00123259 | 0.133837753 | 108.582569 | 1 |
| GRB7 | 4 | 298 | 0.004930359 | 0.531782005 | 107.8586852 | 1 |
| MUC4 | 3 | 223 | 0.003697769 | 0.397944252 | 107.6173906 | 1 |
| SFN | 1 | 74 | 0.00123259 | 0.13205325 | 107.1348014 | 1 |
| TSPEAR | 1 | 68 | 0.00123259 | 0.121346229 | 98.44819587 | 1 |
| LOC100128770 | 1 | 67 | 0.00123259 | 0.119561726 | 97.00042828 | 1 |
| CPA4 | 2 | 131 | 0.002465179 | 0.233769942 | 94.8287769 | 1 |
| GPX2 | 1 | 64 | 0.00123259 | 0.114208216 | 92.65712552 | 1 |
| ADGRF4 | 1 | 62 | 0.00123259 | 0.110639209 | 89.76159035 | 1 |
| C2orf66 | 1 | 60 | 0.00123259 | 0.107070202 | 86.86605518 | 1 |
| ANKRD1 | 19 | 1119 | 0.023419204 | 1.996859274 | 85.265891 | 1 |
| LINC00678 | 2 | 117 | 0.002465179 | 0.208786895 | 84.6944038 | 1 |
| SLC13A2 | 1 | 58 | 0.00123259 | 0.103501196 | 83.97052 | 1 |
| TMEM171 | 1 | 58 | 0.00123259 | 0.103501196 | 83.97052 | 1 |
| LHX1-DT | 1 | 57 | 0.00123259 | 0.101716692 | 82.52275242 | 1 |
| ACE2 | 1 | 56 | 0.00123259 | 0.099932189 | 81.07498483 | 1 |
| TMPRSS2 | 2 | 111 | 0.002465179 | 0.198079874 | 80.35110104 | 1 |
| LINC00261 | 19 | 1030 | 0.023419204 | 1.838038474 | 78.48424284 | 1 |
| LINC02085 | 1 | 54 | 0.00123259 | 0.096363182 | 78.17944966 | 1 |
| KRT15 | 1 | 53 | 0.00123259 | 0.094578679 | 76.73168207 | 1 |
| LOC109864269 | 1 | 53 | 0.00123259 | 0.094578679 | 76.73168207 | 1 |
| TRIM55 | 4 | 211 | 0.004930359 | 0.376530212 | 76.36974018 | 1 |
| IRF4 | 10 | 523 | 0.012325897 | 0.933295264 | 75.71824476 | 1 |
| CALCR | 1 | 52 | 0.00123259 | 0.092794175 | 75.28391449 | 1 |
| OVOL2 | 2 | 104 | 0.002465179 | 0.185588351 | 75.28391449 | 1 |
| SCNN1A | 8 | 405 | 0.009860717 | 0.722723866 | 73.29323406 | 1 |
| SLCO2A1 | 19 | 957 | 0.023419204 | 1.707769728 | 72.92176737 | 1 |
| GCM1 | 1 | 49 | 0.00123259 | 0.087440665 | 70.94061173 | 1 |
| XIST | 3 | 147 | 0.003697769 | 0.262321996 | 70.94061173 | 1 |
| TMEM95 | 1 | 47 | 0.00123259 | 0.083871659 | 68.04507656 | 1 |
| SCN7A | 25 | 1174 | 0.030814742 | 2.09500696 | 67.98716585 | 1 |
| ATP6V0D2 | 1 | 46 | 0.00123259 | 0.082087155 | 66.59730897 | 1 |
| FOXA2 | 17 | 761 | 0.020954024 | 1.358007067 | 64.80889019 | 1 |
| TRPV6 | 4 | 178 | 0.004930359 | 0.3176416 | 64.42565759 | 1 |
| KCNV1 | 3 | 132 | 0.003697769 | 0.235554445 | 63.7017738 | 1 |
| XIRP1 | 6 | 260 | 0.007395538 | 0.463970877 | 62.73659541 | 1 |
| OVCH1 | 1 | 42 | 0.00123259 | 0.074949142 | 60.80623862 | 1 |
| LOC101928841 | 3 | 124 | 0.003697769 | 0.221278418 | 59.84106023 | 1 |
| C8orf49 | 2 | 82 | 0.002465179 | 0.146329277 | 59.35847104 | 1 |
| HSPB8 | 48 | 1958 | 0.059164304 | 3.494057604 | 59.05685279 | 1 |
| NECTIN4 | 5 | 202 | 0.006162948 | 0.360469681 | 58.48981049 | 1 |
| IL11 | 22 | 882 | 0.027116973 | 1.573931975 | 58.04231869 | 1 |
| LOC101928371 | 1 | 40 | 0.00123259 | 0.071380135 | 57.91070345 | 1 |
| EPGN | 2 | 79 | 0.002465179 | 0.140975766 | 57.18681966 | 1 |
| C9orf24 | 1 | 39 | 0.00123259 | 0.069595632 | 56.46293586 | 1 |
| MYRFL | 1 | 39 | 0.00123259 | 0.069595632 | 56.46293586 | 1 |
| FAR2P1 | 6 | 233 | 0.007395538 | 0.415789286 | 56.22164127 | 1 |
| FAM71E2 | 9 | 344 | 0.011093307 | 0.61386916 | 55.33689441 | 1 |
| ADAMTSL2 | 18 | 680 | 0.022186614 | 1.213462293 | 54.69344215 | 1 |
| CDH16 | 1 | 37 | 0.00123259 | 0.066026625 | 53.56740069 | 1 |
| ZNF705A | 1 | 37 | 0.00123259 | 0.066026625 | 53.56740069 | 1 |
| FAT2 | 1 | 36 | 0.00123259 | 0.064242121 | 52.11963311 | 1 |
| TRPA1 | 1 | 36 | 0.00123259 | 0.064242121 | 52.11963311 | 1 |
| FOXI3 | 10 | 358 | 0.012325897 | 0.638852207 | 51.83007959 | 1 |
| SOX21 | 3 | 106 | 0.003697769 | 0.189157358 | 51.15445472 | 1 |
| RAB17 | 11 | 385 | 0.013558486 | 0.687033798 | 50.67186552 | 1 |
| GATA4 | 49 | 1711 | 0.060396894 | 3.053285271 | 50.55368041 | 1 |
| CAPN13 | 3 | 104 | 0.003697769 | 0.185588351 | 50.18927632 | 1 |
| CCL20 | 4 | 134 | 0.004930359 | 0.239123452 | 48.50021414 | 1 |
| POTEE | 2 | 67 | 0.002465179 | 0.119561726 | 48.50021414 | 1 |
| MUC19 | 33 | 1105 | 0.040675459 | 1.971876227 | 48.47827827 | 1 |
| KDF1 | 2 | 66 | 0.002465179 | 0.117777223 | 47.77633035 | 1 |
| CYP26A1 | 16 | 527 | 0.019721435 | 0.940433277 | 47.68584487 | 1 |
| LAMA3 | 4 | 131 | 0.004930359 | 0.233769942 | 47.41438845 | 1 |
| BHLHA15 | 2 | 65 | 0.002465179 | 0.115992719 | 47.05244655 | 1 |
| LNCSRLR | 4 | 130 | 0.004930359 | 0.231985438 | 47.05244655 | 1 |
| PLA2G4C | 15 | 485 | 0.018488845 | 0.865484136 | 46.81115196 | 1 |
| IL31RA | 1 | 32 | 0.00123259 | 0.057104108 | 46.32856276 | 1 |
| BAAT | 7 | 223 | 0.008628128 | 0.397944252 | 46.12173882 | 1 |
| ELF3 | 20 | 631 | 0.024651793 | 1.126021628 | 45.67706735 | 1 |
| NLRP7 | 2 | 63 | 0.002465179 | 0.112423712 | 45.60467897 | 1 |
| PLA1A | 2 | 63 | 0.002465179 | 0.112423712 | 45.60467897 | 1 |
| HNF1A-AS1 | 3 | 94 | 0.003697769 | 0.167743317 | 45.36338437 | 1 |
| KCNK12 | 3 | 94 | 0.003697769 | 0.167743317 | 45.36338437 | 1 |
| ARHGEF38 | 2 | 62 | 0.002465179 | 0.110639209 | 44.88079517 | 1 |
| SBSN | 1 | 31 | 0.00123259 | 0.055319605 | 44.88079517 | 1 |
| HNF1B | 14 | 427 | 0.017256255 | 0.76198294 | 44.15691138 | 1 |

TABLE 6
Top 100 downregulated genes in response to CSNK2B-KD in CMS cells

| | Control/ | CSNK2B- | Control/ | CSNK2B/ | | E- |
|---|---|---|---|---|---|---|
| Gene_Symbol | Count | KD/Count | Value | Value | Fold_Change | FDR |
| HEMGN | 5086 | 0 | 6.26895107 | 0 | >10000 down | 1 |
| EPX | 4185 | 0 | 5.15838777 | 0 | >10000 down | 1 |
| GATA1 | 2794 | 0 | 3.44385554 | 0 | >10000 down | 1 |
| S100A9 | 2752 | 0 | 3.39208677 | 0 | >10000 down | 1 |
| S100A8 | 1982 | 0 | 2.44299273 | 0 | >10000 down | 1 |
| GYPB | 1906 | 0 | 2.34931591 | 0 | >10000 down | 1 |
| FCN1 | 1585 | 0 | 1.95365463 | 0 | >10000 down | 1 |
| AHSP | 1509 | 0 | 1.85997781 | 0 | >10000 down | 1 |
| RNASE2 | 1440 | 0 | 1.77492913 | 0 | >10000 down | 1 |
| CA1 | 1297 | 0 | 1.5986688 | 0 | >10000 down | 1 |
| C17orf99 | 1043 | 0 | 1.28559103 | 0 | >10000 down | 1 |
| BPI | 911 | 0 | 1.12288919 | 0 | >10000 down | 1 |
| GYPA | 9068 | 1 | 11.1771231 | 0.0017845 | −6263.4363 | 1 |
| ALAS2 | 21872 | 3 | 26.9592013 | 0.00535351 | −5035.7991 | 1 |
| FAM83A | 6695 | 1 | 8.25218785 | 0.0017845 | −4624.361 | 1 |
| MPO | 34135 | 7 | 42.0744484 | 0.01249152 | −3368.2399 | 1 |
| HBZ | 3536 | 1 | 4.35843708 | 0.0017845 | −2442.381 | 1 |
| OLFM4 | 2360 | 1 | 2.90891162 | 0.0017845 | −1630.0959 | 1 |
| C11orf21 | 2073 | 1 | 2.55515839 | 0.0017845 | −1431.8597 | 1 |
| SRGN | 5803 | 3 | 7.15271786 | 0.00535351 | −1336.08 | 1 |
| SLC4A1 | 46639 | 25 | 57.4867497 | 0.04461258 | −1288.577 | 1 |
| EPB42 | 1738 | 1 | 2.14224085 | 0.0017845 | −1200.4689 | 1 |
| GFI1B | 3018 | 2 | 3.71995563 | 0.00356901 | −1042.2944 | 1 |
| CLC | 1221 | 1 | 1.50499199 | 0.0017845 | −843.36741 | 1 |
| PKLR | 10497 | 9 | 12.9384938 | 0.01606053 | −805.60813 | 1 |
| SPTA1 | 11459 | 10 | 14.124245 | 0.01784503 | −791.49444 | 1 |
| HBE1 | 1138 | 1 | 1.40268705 | 0.0017845 | −786.03777 | 1 |
| PRTN3 | 9500 | 9 | 11.7096019 | 0.01606053 | −729.09186 | 1 |
| NT5DC4 | 803 | 1 | 0.98976951 | 0.0017845 | −554.64704 | 1 |
| PRG3 | 786 | 1 | 0.96881548 | 0.0017845 | −542.90482 | 1 |
| MNDA | 778 | 1 | 0.95895476 | 0.0017845 | −537.37907 | 1 |
| AHSG | 2067 | 3 | 2.54776285 | 0.00535351 | −475.90512 | 1 |
| GYPE | 2434 | 4 | 3.00012326 | 0.00713801 | −420.30227 | 1 |
| TSPAN32 | 1164 | 2 | 1.43473438 | 0.00356901 | −401.99823 | 1 |
| PTPN7 | 495 | 1 | 0.61013189 | 0.0017845 | −341.90571 | 1 |
| RHAG | 4424 | 9 | 5.4529767 | 0.01606053 | −339.52657 | 1 |
| CYBB | 1886 | 4 | 2.32466412 | 0.00713801 | −325.67382 | 1 |
| RLN3 | 469 | 1 | 0.57808456 | 0.0017845 | −323.94702 | 1 |
| KLF1 | 3961 | 9 | 4.88228769 | 0.01606053 | −303.99293 | 1 |
| TMPRSS4 | 402 | 1 | 0.49550105 | 0.0017845 | −277.66888 | 1 |
| SPTB | 15691 | 40 | 19.3405645 | 0.07138013 | −270.95164 | 1 |
| XK | 4251 | 11 | 5.23973869 | 0.01962954 | −266.93134 | 1 |
| TUBB1 | 5054 | 14 | 6.2295082 | 0.02498305 | −249.34941 | 1 |
| SLFN14 | 358 | 1 | 0.4412671 | 0.0017845 | −247.27726 | 1 |
| LYZ | 3829 | 11 | 4.71958585 | 0.01962954 | −240.43287 | 1 |
| HK3 | 341 | 1 | 0.42031308 | 0.0017845 | −235.53504 | 1 |
| IKZF1 | 4600 | 14 | 5.66991249 | 0.02498305 | −226.9504 | 1 |
| MLC1 | 606 | 2 | 0.74694934 | 0.00356901 | −209.28774 | 1 |
| NFE2 | 5696 | 19 | 7.02083077 | 0.03390556 | −207.07017 | 1 |
| P2RY8 | 282 | 1 | 0.34759029 | 0.0017845 | −194.78265 | 1 |
| TAL1 | 10396 | 37 | 12.8140022 | 0.06602662 | −194.07326 | 1 |
| MS4A3 | 1113 | 4 | 1.3718723 | 0.00713801 | −192.19245 | 1 |
| IL1RN | 258 | 1 | 0.31800814 | 0.0017845 | −178.2054 | 1 |
| PTPRC | 493 | 2 | 0.60766671 | 0.00356901 | −170.26213 | 1 |
| PLEK | 973 | 4 | 1.19930975 | 0.00713801 | −168.0173 | 1 |
| RETN | 239 | 1 | 0.29458893 | 0.0017845 | −165.08175 | 1 |
| SELP | 190 | 1 | 0.23419204 | 0.0017845 | −131.23653 | 1 |
| CR1L | 558 | 3 | 0.68778504 | 0.00535351 | −128.47366 | 1 |
| TESC | 1432 | 8 | 1.76506841 | 0.01427603 | −123.63863 | 1 |
| SLC30A10 | 4253 | 24 | 5.24220387 | 0.04282808 | −122.40109 | 1 |
| NEUROG1 | 175 | 1 | 0.21570319 | 0.0017845 | −120.87575 | 1 |
| CSF2RB | 835 | 5 | 1.02921238 | 0.00892252 | −115.35001 | 1 |
| ELANE | 321 | 2 | 0.39566128 | 0.00356901 | −110.86034 | 1 |
| LXN | 2247 | 14 | 2.76962899 | 0.02498305 | −110.86034 | 1 |
| SERPINA1 | 1589 | 10 | 1.95858499 | 0.01784503 | −109.75519 | 1 |
| LOC100129129 | 314 | 2 | 0.38703316 | 0.00356901 | −108.44282 | 1 |
| CD53 | 152 | 1 | 0.18735363 | 0.0017845 | −104.98923 | 1 |
| MYL4 | 1587 | 11 | 1.95611981 | 0.01962954 | −99.651856 | 1 |
| ST6GALNAC1 | 1291 | 9 | 1.59127327 | 0.01606053 | −99.079746 | 1 |
| PRG2 | 4062 | 29 | 5.00677924 | 0.0517506 | −96.74824 | 1 |
| ANK1 | 38046 | 276 | 46.8951066 | 0.49252293 | −95.214057 | 1 |
| PADI4 | 684 | 5 | 0.84309133 | 0.00892252 | −94.490304 | 1 |
| DEFA8P | 136 | 1 | 0.1676322 | 0.0017845 | −93.93773 | 1 |
| LGALS12 | 134 | 1 | 0.16516702 | 0.0017845 | −92.556292 | 1 |
| SPN | 7044 | 54 | 8.68236164 | 0.09636318 | −90.100404 | 1 |
| OR2AT4 | 128 | 1 | 0.15777148 | 0.0017845 | −88.411981 | 1 |
| TYROBP | 376 | 3 | 0.46345372 | 0.00535351 | −86.570065 | 1 |
| HBM | 122 | 1 | 0.15037594 | 0.0017845 | −84.267669 | 1 |
| FGG | 1791 | 15 | 2.2075681 | 0.02676755 | −82.471801 | 1 |
| TREML2 | 706 | 6 | 0.87020831 | 0.01070702 | −81.274555 | 1 |
| SPI1 | 939 | 8 | 1.1574017 | 0.01427603 | −81.073096 | 1 |
| F13A1 | 117 | 1 | 0.14421299 | 0.0017845 | −80.814076 | 1 |
| APOC3 | 348 | 3 | 0.42894121 | 0.00535351 | −80.123358 | 1 |
| FYB1 | 342 | 3 | 0.42154567 | 0.00535351 | −78.74192 | 1 |
| LTF | 226 | 2 | 0.27856527 | 0.00356901 | −78.051202 | 1 |
| TAGAP | 225 | 2 | 0.27733268 | 0.00356901 | −77.705842 | 1 |
| ITGAL | 438 | 4 | 0.53987428 | 0.00713801 | −75.633687 | 1 |
| MYO1F | 1082 | 10 | 1.33366202 | 0.01784503 | −74.735752 | 1 |
| LCP2 | 423 | 4 | 0.52138543 | 0.00713801 | −73.043492 | 1 |
| PROK2 | 105 | 1 | 0.12942192 | 0.0017845 | −72.525453 | 1 |
| CTCFL | 104 | 1 | 0.12818933 | 0.0017845 | −71.834734 | 1 |
| AQP2 | 103 | 1 | 0.12695674 | 0.0017845 | −71.144016 | 1 |
| LILRA2 | 100 | 1 | 0.12325897 | 0.0017845 | −69.07186 | 1 |
| FCGR2A | 199 | 2 | 0.24528534 | 0.00356901 | −68.726501 | 1 |
| GRAP2 | 893 | 9 | 1.10070258 | 0.01606053 | −68.534634 | 1 |
| PSTPIP1 | 196 | 2 | 0.24158758 | 0.00356901 | −67.690423 | 1 |
| ITGB3 | 2595 | 27 | 3.1985702 | 0.04818159 | −66.385732 | 1 |
| EVI2A | 96 | 1 | 0.11832861 | 0.0017845 | −66.308986 | 1 |
| HBQ1 | 187 | 2 | 0.23049427 | 0.00356901 | −64.582189 | 1 |
| P2RY13 | 91 | 1 | 0.11216566 | 0.0017845 | −62.855393 | 1 |

### Example 8—Phosphorylation Targets of CSNK2B

Proteomics analysis was conducted on CSNK2B-KD CMS cells and control CMS cells. CSNK2B regulates the phosphorylation of erythropoietic regulators, and since inhibition of Casein kinase 2 activity suppresses erythropoiesis, its potential phosphorylation targets were identified in early stage hematopoietic (CD34+) cells following CSNK2B KD using phospho-proteomics. Using 20-50 μg of protein, a total number of ~4600 specific phospho-peptides were identified within ~1800 proteins in the wildtype and KD cells. Among them, phosphorylation of 9 proteins were significantly altered (|FC|>2, p<0.05, an improved analytical pipeline. Noteworthy. 4 of these 9 proteins were previously characterized as erythropoietic regulators (e.g., ZC3H11TB, ARHGEF2, MARCKS and SPTBN1), suggesting that CSNK2B/CK2 modifies specific erythropoietic regulators in the erythroid lineage.

Table 7 lists the protein targets that were phosphorylated by CSNK2B, and these protein candidates of interest for future studies.

TABLE 7
Phosphorylation targets of Csnk2B

| | | Phosphopeptide/ | | p- | |
|---|---|---|---|---|---|
| protein_ID | Gene_Symbol | Phosporylated AA | Fold_Change | value | Name |
| A0A1B0GTU1 | ZC3H11B | V(+229.16)QQSSESST(+79.97) | −3.00 | 0.022 | Zinc finger CCCH domain-containing protein |
| | | SSPSQHEATPGAR | | | 11B |
| A0A0A0MSM0 | HSPH1 | I(+229.16)ES(+79.97)PK(+229.16)LER | 2.00 | 0.019 | Heat shock protein 105 kDa |
| A0A804HL01 | ADD1 | F(+229.16)RTPS(+79.97)FLK | 2.01 | 0.046 | Alpha-adducin |
| | | (+229.16)K(+229.16) | | | |
| Q9UQ35 | SRRM2 | A(+229.16)RS(+79.97)RT | 2.01 | 0.007 | Serine/arginine repetitive matrix protein 2 (300 |
| | | (+79.97)PPSAPSQSR | | | kDa nuclear matrix antigen) (Serine/arginine- |
| | | | | | rich splicing factor-related nuclear matrix |
| | | | | | protein of 300 kDa) (SR-related nuclear matrix |
| | | | | | protein of 300 kDa) (Ser/Arg-related nuclear |
| | | | | | matrix protein of 300 kDa) (Splicing |
| | | | | | coactivator subunit SRm300) (Tax-responsive |
| | | | | | enhancer element-binding protein 803) |
| | | | | | (TaxREB803) |
| Q99959 | PKP2 | G(+229.16)TAQYSSQK(+229.16) | 2.01 | 0.026 | Plakophilin-2 |
| | | S(+79.97)VEER | | | |
| Q9H2D6 | TARA | A(+229.16)S(+79.97)SPNRTTQQDSPR | 2.06 | 0.009 | TRIO and F-actin-binding protein (Protein |
| | | | | | Tara) (Trio-associated repeat on actin) |
| P10412 | H14 | G(+229.16)TGASGS(+79.97)FK | 2.07 | 0.050 | Histone H1.4 (Histone H1b) (Histone H1s-4) |
| | | (+229.16)LNK(+229.16)K(+229.16) | | | |
| P16402 | H13 | G(+229.16)TGASGS(+79.97)FK | 2.07 | 0.050 | Histone H1.3 (Histone H1c) (Histone H1s-2) |
| | | (+229.16)LNK(+229.16)K(+229.16) | | | |
| P16403 | H12 | G(+229.16)TGASGS(+79.97)FK | 2.07 | 0.050 | Histone H1.2 (Histone H1c) (Histone H1d) |
| | | (+229.16)LNK(+229.16)K(+229.16) | | | (Histone H1s-1) |
| A0A087WUZ3 | SPTBN1 | R(+229.16)PPS(+79.97)PEPSTK | 2.14 | 0.016 | Spectrin beta chain |
| | | (+229.16) | | | |
| Q01518 | CAP1 | S(+229.16)GPK(+229.16)PFSAPK | 2.17 | 0.026 | Adenylyl cyclase-associated protein 1 (CAP 1) |
| | | (+229.16)PQTS(+79.97)PSPK | | | |
| | | (+229.16) | | | |
| | | S(+229.16)GPK(+229.16)PFSAPK | 2.03 | 0.014 | |
| | | (+229.16)PQTS(+79.97)PSPK | | | |
| | | (+229.16)R | | | |
| A0A5F9ZI21 | ARHGEF2 | S(+229.16)ES(+79.97)LESPRGER | 2.22 | 0.017 | Rho guanine nucleotide exchange factor 2 |
| | | | | | (Guanine nucleotide exchange factor H1) |
| A0A494C050 | NKAP | A(+229.16)PVSGSRS(+79.97) | 2.22 | 0.011 | NF-kappa-B-activating protein (Fragment) |
| | | PDREASGSGGR | | | |
| P49750 | YLPM1 | R(+229.16)AGS(+79.97)QERGPLR | 2.40 | 0.010 | YLP motif-containing protein 1 (Nuclear |
| | | | | | protein ZAP3) (ZAP113) |
| H3BNU9 | CARHSP1 | T(+229.16)FS(+79.97)ATVR | 2.78 | 0.032 | Calcium-regulated heat-stable protein 1 |
| | | | | | (Fragment) |
| P49006 | MRP | L(+229.16)SGLS(+79.97)FK(+229.16)R | 3.05 | 0.037 | MARCKS-related protein (MARCKS-like |
| | | | | | protein 1) (Macrophage myristoylated alanine- |
| | | | | | rich C kinase substrate) (Mac-MARCKS) |
| | | | | | (MacMARCKS) |
| P29966 | MARCS | L(+229.16)SGFS(+79.97)FK | 3.26 | 0.035 | Myristoylated alanine-rich C-kinase substrate |
| | | (+229.16)K(+229.16) | | | (MARCKS) (Protein kinase C substrate, 80 |
| | | S(+229.16)FK(+229.16)LSGFS | 3.16 | 0.034 | kDa protein, light chain) (80K-L protein) |
| | | (+79.97)FK(+229.16)K(+229.16) | | | (PKCSL) |

### B. LINC00431

### Example 9—LINC00431 and Downstream Target Genes

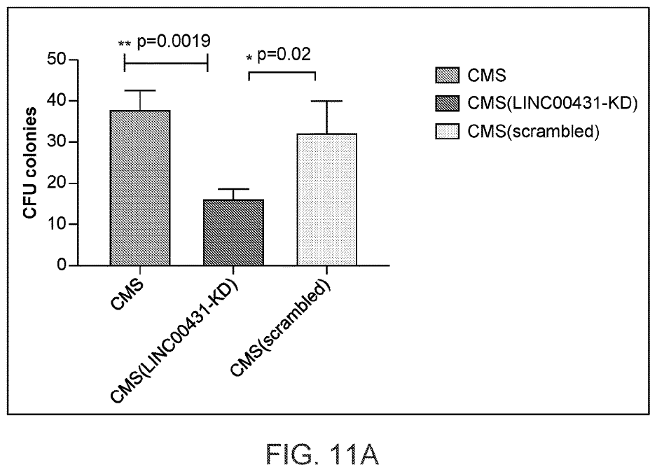

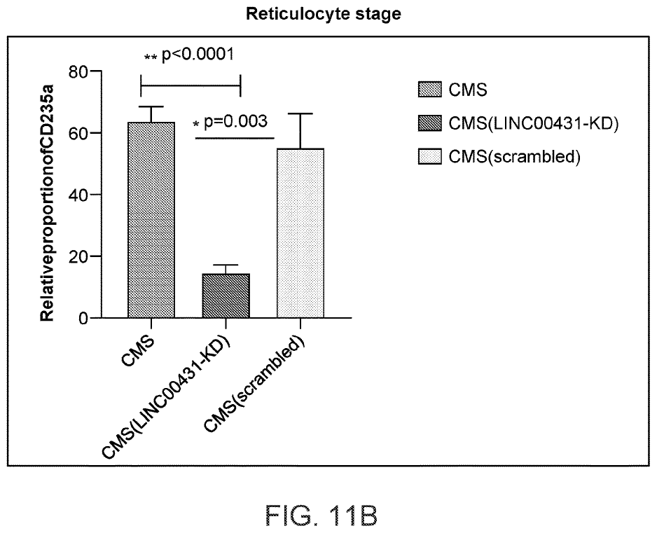

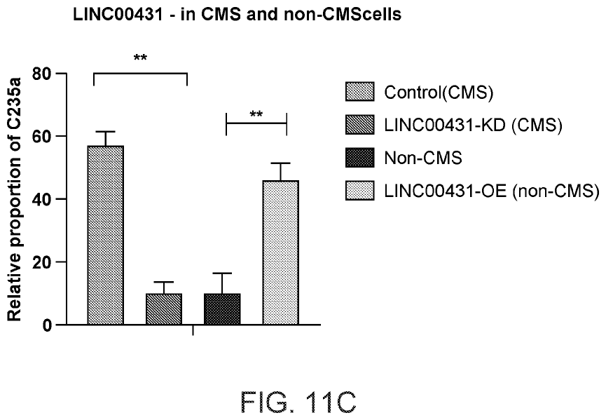

In order to assess the functional role of the candidate nuclear lncRNAs LINC00431 that was downregulated in non-CMS cells (FIG. 8), colony-forming assay was performed among CMS cells, LINC00431-KD CMS cells and scrambled CMS cells. As shown in FIGS. 11A-11B, KD of LINC00431 in CMS cells led to a significant reduction of colonies in the later erythroid stages such as the CFU-E and reticulocyte stages (CD71+ and/or CD235a+ cells). Particularly, KD of LINC00431 resulted in a major reduction (from ~60% to ~7%, p<0.0001) of CD235a+ erythroid cells (Reticulocyte stage-a later erythroid stage. FIG. 11B). Moreover, FIG. 11C showed that the reduction in CD235a+ erythroid cells observed in LINC00431-KD CMS cells is comparable to the levels in non-CMS cells. The level of CD235a+ erythroid cells in non-CMS cells was effectively reversed by OE of LINC00431 in non-CMS cells. The results together suggested that LINC00431 plays an important role in reticulocyte stage in both CMS and non-CMS cells.

To further explore the downstream target genes of LINC00431, RNA-seq was performed using LINC00431-KD CMS cells and control CMS cells under hypoxia condition. Among the identified genes exhibiting at least a 2-fold change with statistical significance, 20 genes were upregulated, and 93 genes were downregulated. The total 113 gene candidates identified by RNA-seq in LINC00431-KD CMS cells vs. control CMS cells are shown in the table below.

TABLE 8
Genes upregulated or downregulated in response to LINC00431-KD in CMS cells

| Gene_Symbol | baseMean | log2FoldChange | lfcSE | stat | pvalue | padj |
|---|---|---|---|---|---|---|
| CNPY1 | 55.73948011 | 6.356734711 | 2.890682849 | 2.199042594 | 0.027874894 | 0.998885201 |
| ZNF676 | 133.3593212 | 6.025864465 | 2.358998575 | 2.554416323 | 0.010636599 | 0.998885201 |
| TMEM132C | 128.8943824 | 5.975968483 | 2.357652674 | 2.534711134 | 0.011254009 | 0.998885201 |
| ZNF667-AS1 | 134.4033011 | 5.614914658 | 2.248870977 | 2.496770475 | 0.012533005 | 0.998885201 |
| ZNF667 | 109.0297266 | 4.975135494 | 2.151169715 | 2.312758245 | 0.020735935 | 0.998885201 |
| ZFP28 | 59.98765463 | 4.414105587 | 2.21558832 | 1.992295025 | 0.046338697 | 0.998885201 |
| CHL1 | 86.18844486 | 4.114757914 | 1.997270774 | 2.060190319 | 0.03938035 | 0.998885201 |
| NELL1 | 103.9036905 | 4.016322014 | 1.912611788 | 2.099914912 | 0.035736327 | 0.998885201 |
| KIAA0125 | 97.95043879 | 3.925770955 | 1.905338946 | 2.060405558 | 0.039359786 | 0.998885201 |
| PPAP2C | 122.2185831 | 3.699557931 | 1.782046144 | 2.076016911 | 0.037892387 | 0.998885201 |
| EN2 | 96.31761634 | 3.588767466 | 1.817131212 | 1.974963306 | 0.048272312 | 0.998885201 |
| GPR55 | 135.3243804 | 3.439250723 | 1.68510739 | 2.040968275 | 0.041253981 | 0.998885201 |
| EOMES | 142.1771026 | 3.260037169 | 1.623633928 | 2.007864651 | 0.044657676 | 0.998885201 |
| CER1 | 354.3481307 | 2.961550102 | 1.377313076 | 2.150237411 | 0.03153644 | 0.998885201 |
| CRABP1 | 467.5031311 | 2.944354926 | 1.338718708 | 2.199382819 | 0.027850713 | 0.998885201 |
| NEFM | 315.3629755 | 2.663043427 | 1.308411627 | 2.035325407 | 0.041818141 | 0.998885201 |
| NODAL | 451.4024754 | 2.481947148 | 1.210626046 | 2.050135263 | 0.040351233 | 0.998885201 |
| LFNG | 403.0323052 | 2.287098369 | 1.164927673 | 1.963296453 | 0.049611737 | 0.998885201 |
| CHCHD2 | 2299.69468 | 1.701214265 | 0.867830608 | 1.960306826 | 0.049959939 | 0.998885201 |
| APLNR | 4204.625835 | 1.65018725 | 0.839393014 | 1.965929216 | 0.049306788 | 0.998885201 |
| UBC | 40070.75591 | −1.640414928 | 0.820908186 | −1.998292812 | 0.045684924 | 0.998885201 |
| SLC2A1 | 15019.68133 | −1.732837025 | 0.853433216 | −2.030430727 | 0.042312776 | 0.998885201 |
| SERPINB9 | 9199.171056 | −1.777204725 | 0.870643339 | −2.041254605 | 0.041225527 | 0.998885201 |
| TUBB2B | 4098.305999 | −1.779990831 | 0.881196123 | −2.019971247 | 0.04338637 | 0.998885201 |
| EPAS1 | 4299.233647 | −1.840583901 | 0.899504254 | −2.04622034 | 0.040734697 | 0.998885201 |
| NBPF1 | 1397.348918 | −1.899131556 | 0.949961502 | −1.999166864 | 0.045590303 | 0.998885201 |
| CBWD2 | 806.0524554 | −1.972153534 | 1.003723051 | −1.96483834 | 0.049432951 | 0.998885201 |
| TUBB2A | 3611.908587 | −2.004166043 | 0.9533297 | −2.102280086 | 0.035528748 | 0.998885201 |
| AQP1 | 3077.857475 | −2.064541496 | 0.975165904 | −2.117118214 | 0.034249816 | 0.998885201 |
| EDARADD | 584.0534299 | −2.123455812 | 1.076053729 | −1.973373406 | 0.048453035 | 0.998885201 |
| LOC399491 | 976.0003698 | −2.173361741 | 1.053335125 | −2.063314599 | 0.039082748 | 0.998885201 |
| LOC285074 | 479.9120415 | −2.219428434 | 1.124189933 | −1.974246849 | 0.048353681 | 0.998885201 |
| ZNF322 | 750.1866983 | −2.327266359 | 1.11738015 | −2.082788351 | 0.03727052 | 0.998885201 |
| PKD1P6 | 614.9102626 | −2.337317845 | 1.136415553 | −2.056745737 | 0.03971069 | 0.998885201 |
| CCBE1 | 560.4463828 | −2.371057377 | 1.15482527 | −2.053174138 | 0.040055692 | 0.998885201 |
| PDXDC2P | 611.2941484 | −2.561454508 | 1.202826951 | −2.129528695 | 0.033210543 | 0.998885201 |
| NSUN5P1 | 273.9339888 | −2.571808 | 1.299645533 | −1.978853414 | 0.047832513 | 0.998885201 |
| MET | 266.698384 | −2.608061576 | 1.314324296 | −1.984336425 | 0.047218348 | 0.998885201 |
| GRHL2 | 344.3175498 | −2.74665441 | 1.318383277 | −2.083350462 | 0.03721929 | 0.998885201 |
| COL12A1 | 297.6889218 | −2.909080501 | 1.384292384 | −2.101492817 | 0.035597728 | 0.998885201 |
| GSTA1 | 420.8312853 | −2.982901186 | 1.35964901 | −2.193875894 | 0.028244333 | 0.998885201 |
| AGT | 283.2177121 | −3.01107357 | 1.420150323 | −2.120249893 | 0.033984977 | 0.998885201 |
| MGC57346 | 206.1259385 | −3.014663513 | 1.473652289 | −2.045708839 | 0.040785026 | 0.998885201 |
| ARHGAP11B | 144.9322372 | −3.087547817 | 1.563859004 | −1.974313419 | 0.048346116 | 0.998885201 |
| APOC3 | 176.8006366 | −3.120576264 | 1.532016388 | −2.036907887 | 0.041659274 | 0.998885201 |
| UGT2B11 | 429.4431069 | −3.320792723 | 1.447325479 | −2.294433955 | 0.021765585 | 0.998885201 |
| MUC6 | 110.0872121 | −3.437602895 | 1.726049569 | −1.991601491 | 0.0464148 | 0.998885201 |
| PDCD6IPP2 | 111.6585842 | −3.659994071 | 1.782473457 | −2.053323182 | 0.040041244 | 0.998885201 |
| PLG | 176.1503437 | −3.835081309 | 1.721984289 | −2.227129094 | 0.025938647 | 0.998885201 |
| KRT16P1 | 85.53747678 | −3.840563965 | 1.904666924 | −2.016396629 | 0.043758518 | 0.998885201 |
| C9orf129 | 71.65510962 | −3.911528998 | 1.979273763 | −1.976244555 | 0.048127086 | 0.998885201 |
| SULT1E1 | 160.8519184 | −3.965980073 | 1.774347015 | −2.235177245 | 0.025405715 | 0.998885201 |
| LOC391322 | 69.93922797 | −4.007912586 | 2.013172745 | −1.990843854 | 0.046498056 | 0.998885201 |
| TDGF1P3 | 57.40066423 | −4.192481278 | 2.132919137 | −1.965607231 | 0.049343998 | 0.998885201 |
| DDX11L2 | 93.52804252 | −4.196588895 | 1.971421575 | −2.128712067 | 0.03327809 | 0.998885201 |
| LOC388436 | 59.04429112 | −4.437591461 | 2.188102582 | −2.02805458 | 0.042554678 | 0.998885201 |
| LOC79999 | 59.04429112 | −4.437591461 | 2.188102582 | −2.02805458 | 0.042554678 | 0.998885201 |
| SPDYE6 | 67.47918986 | −4.437592583 | 2.13910213 | −2.074511787 | 0.038031805 | 0.998885201 |
| MAP2K4P1 | 46.20590391 | −4.575090898 | 2.325027393 | −1.967757847 | 0.049095906 | 0.998885201 |
| SPDYE5 | 65.76330821 | −4.599862073 | 2.190996083 | −2.099438748 | 0.035778242 | 0.998885201 |
| MUC3A | 183.7796357 | −4.643625921 | 1.908634982 | −2.432956519 | 0.014976099 | 0.998885201 |
| MUC4 | 187.1391442 | −4.670795999 | 1.910998221 | −2.44416554 | 0.014518763 | 0.998885201 |
| PGM5P2 | 71.81042358 | −4.731773076 | 2.193716382 | −2.15696665 | 0.031008263 | 0.998885201 |
| POUSF1P4 | 41.80241543 | −4.759511884 | 2.421781061 | −1.965294039 | 0.049380215 | 0.998885201 |
| KRT16 | 191.0982997 | −4.868457812 | 1.951639298 | −2.494547952 | 0.012611768 | 0.998885201 |
| DENND5B- | 56.95633124 | −4.888247146 | 2.322287297 | −2.104927824 | 0.035297591 | 0.998885201 |
| AS1 | | | | | | |
| KRT16P3 | 93.01145477 | −4.918716853 | 2.153605537 | −2.283945118 | 0.022374757 | 0.998885201 |
| KRT23 | 72.11024706 | −4.968101992 | 2.253048951 | −2.20505728 | 0.027450075 | 0.998885201 |
| LOC441081 | 40.08653378 | −5.125633586 | 2.547123451 | −2.012322404 | 0.044185962 | 0.998885201 |
| POU5F1P3 | 55.24044959 | −5.174547159 | 2.412233045 | −2.145127383 | 0.031942664 | 0.998885201 |
| GLUD1P7 | 42.77414061 | −5.221848381 | 2.542801466 | −2.053580844 | 0.040016278 | 0.998885201 |
| RIMBP3B | 57.92805643 | −5.244936102 | 2.411137865 | −2.175294983 | 0.029608018 | 0.998885201 |
| RIMBP3C | 59.27185984 | −5.278883247 | 2.410666502 | −2.189802381 | 0.028538572 | 0.998885201 |
| LOC729737 | 141.0885543 | −5.362181374 | 2.137122382 | −2.509066125 | 0.012105082 | 0.998885201 |
| DDX11L1 | 49.4931577 | −5.437575888 | 2.534370207 | −2.145533384 | 0.031910225 | 0.998885201 |
| BCRP2 | 52.85266624 | −5.534436887 | 2.531115166 | −2.186560675 | 0.028774611 | 0.998885201 |
| MUC5B | 52.85266624 | −5.534436887 | 2.531115166 | −2.186560675 | 0.028774611 | 0.998885201 |
| KATNBL1P6 | 36.35494701 | −5.580525293 | 2.736968446 | −2.038943964 | 0.04145562 | 0.998885201 |
| LPA | 37.02684871 | −5.607492194 | 2.735070007 | −2.05021889 | 0.040343075 | 0.998885201 |
| MUC15 | 37.02684871 | −5.607492194 | 2.735070007 | −2.05021889 | 0.040343075 | 0.998885201 |
| CRH | 39.04255384 | −5.68549428 | 2.729745976 | −2.082792439 | 0.037270147 | 0.998885201 |
| SOWAHA | 42.40206238 | −5.806799909 | 2.721925992 | −2.133342319 | 0.032896655 | 0.998885201 |
| SPIN2A | 45.76157092 | −5.918692159 | 2.715450585 | −2.179635377 | 0.0292845 | 0.998885201 |
| INTS4L2 | 70.99401236 | −5.968087609 | 2.519413731 | −2.368839836 | 0.017843979 | 0.998885201 |
| LINC00960 | 115.3395251 | −6.677034582 | 2.511262057 | −2.658836247 | 0.007841106 | 0.998885201 |
| ANAPC1P1 | 91.45088709 | −6.92941258 | 2.674573711 | −2.590847488 | 0.009573991 | 0.998885201 |
| GGTLC2 | 96.82610075 | −7.012467791 | 2.672785896 | −2.623654891 | 0.008699188 | 0.998885201 |
| DDX11L5 | 60.17133027 | −7.328307885 | 3.03160208 | −2.417305336 | 0.015635895 | 0.998885201 |
| C6orf10 | 20.15705125 | −8.20205656 | 4.167956746 | −1.96788428 | 0.049081354 | 0.998885201 |
| CCL3L3 | 20.15705125 | −8.20205656 | 4.167956746 | −1.96788428 | 0.049081354 | 0.998885201 |
| LGALS16 | 20.15705125 | −8.20205656 | 4.167956746 | −1.96788428 | 0.049081354 | 0.998885201 |
| AMY1A | 20.82895296 | −8.249362679 | 4.152701067 | −1.9865053 | 0.046977245 | 0.998885201 |
| AMY1B | 20.82895296 | −8.249362679 | 4.152701067 | −1.9865053 | 0.046977245 | 0.998885201 |
| AMY1C | 20.82895296 | −8.249362679 | 4.152701067 | −1.9865053 | 0.046977245 | 0.998885201 |
| CCL3L1 | 20.82895296 | −8.249362679 | 4.152701067 | −1.9865053 | 0.046977245 | 0.998885201 |
| FAM197Y2 | 20.82895296 | −8.249362679 | 4.152701067 | −1.9865053 | 0.046977245 | 0.998885201 |
| FAM197Y5 | 20.82895296 | −8.249362679 | 4.152701067 | −1.9865053 | 0.046977245 | 0.998885201 |
| LINC00965 | 20.82895296 | −8.249362679 | 4.152701067 | −1.9865053 | 0.046977245 | 0.998885201 |
| LOC286297 | 20.82895296 | 8.249362679 | 4.152701067 | −1.9865053 | 0.046977245 | 0.998885201 |
| TRIM31 | 20.82895296 | −8.249362679 | 4.152701067 | −1.9865053 | 0.046977245 | 0.998885201 |
| HYDIN2 | 22.84465808 | −8.382630213 | 4.111914611 | −2.03861972 | 0.041487996 | 0.998885201 |
| FAM230B | 23.51655979 | −8.424450679 | 4.099569008 | −2.054960085 | 0.03988286 | 0.998885201 |
| AQP7P1 | 24.86036321 | −8.504621513 | 4.07695392 | −2.086023458 | 0.036976498 | 0.998885201 |
| LY6G6C | 28.21987175 | −8.687486537 | 4.028007754 | −2.156770063 | 0.031023585 | 0.998885201 |
| GCM1 | 34.93888883 | −8.995609971 | 3.954121377 | −2.274995913 | 0.022906175 | 0.998885201 |
| RGPD2 | 1374.927659 | −9.263975839 | 4.685171171 | −1.9772972 | 0.048008046 | 0.998885201 |
| NPIPA7 | 551.7035573 | −9.531905988 | 4.862663373 | −1.960223288 | 0.049969698 | 0.998885201 |
| NPIPA8 | 551.7035573 | −9.531905988 | 4.862663373 | −1.960223288 | 0.049969698 | 0.998885201 |
| IER3 | 1209.567584 | −9.66461598 | 4.813207173 | −2.007936835 | 0.044650004 | 0.998885201 |
| ZBTB12 | 704.8971468 | −9.885832428 | 4.948585564 | −1.997708699 | 0.045748251 | 0.998885201 |
| DAXX | 2177.106044 | −10.51328677 | 5.017198255 | −2.095449738 | 0.036131032 | 0.998885201 |
| SKIV2L | 3561.750956 | −15.66659908 | 6.766466554 | −2.31532942 | 0.020594911 | 0.998885201 |

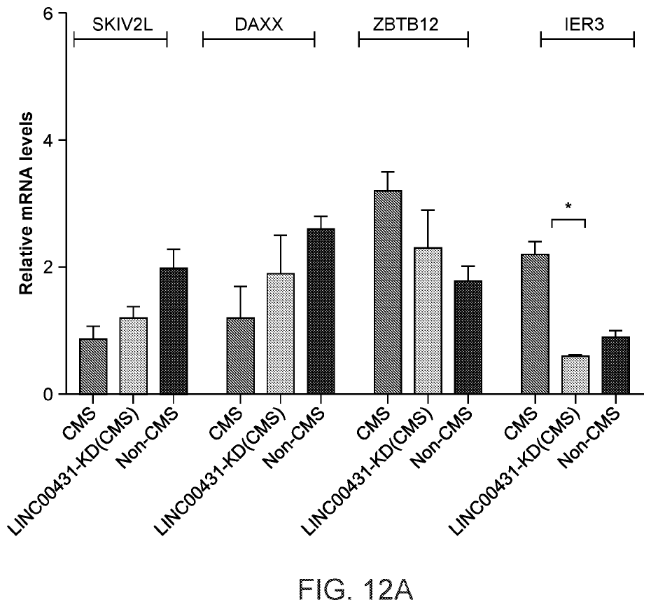

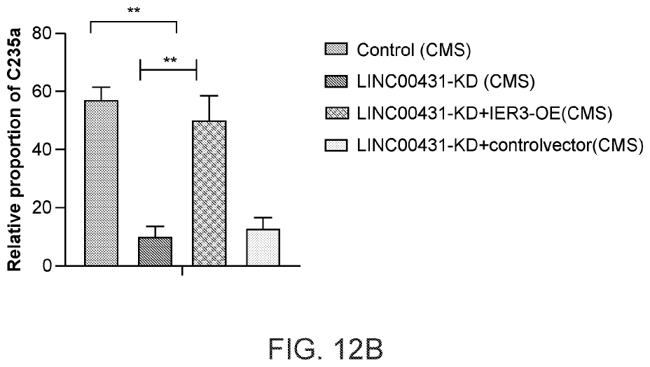

Specifically, among the genes targeted by LINC00431, top 5 upregulated genes included CNPY1, ZF676, TMEM132C, ZNF667-AS1, and ZNF667, and top 6 downregulated genes included SKIV2L. DAXX, ZBTB12, IER3, NPIPA8, and NPIPA7. These gene candidates were further validated by qPCR (FIG. 12A), revealing that among them, only IER3 exhibited a significant reduction in LINC00431-KD CMS cells compared to control CMS cells. In order to assess the functional role of IER3 as a target of LINC00431, IER3 was overexpressed in LINC00431-KD CMS cells. As shown in FIG. 12B, OE of IER3 in LINC00431-KD CMS cells effectively restored the level of CD235a+ erythroid cells to levels comparable to those in control CMS cells.

### C. APOBEC3B-AS1 and APOBEC3B

### Example 10—APOBEC3B-AS1 and Downstream Target Genes

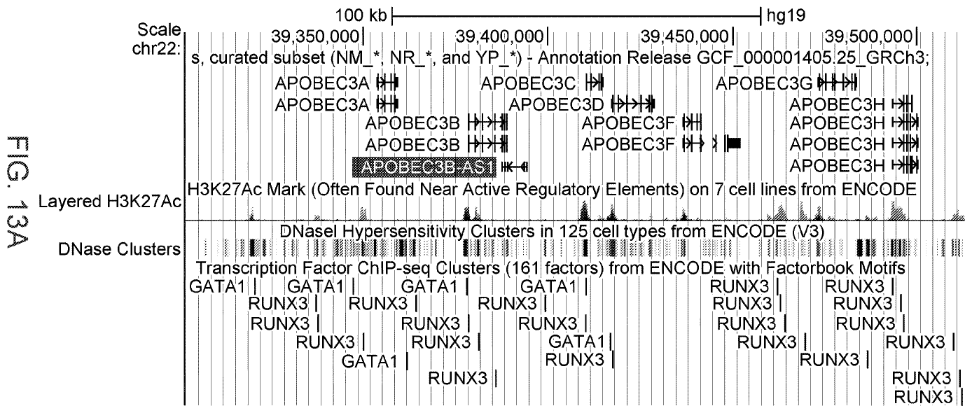

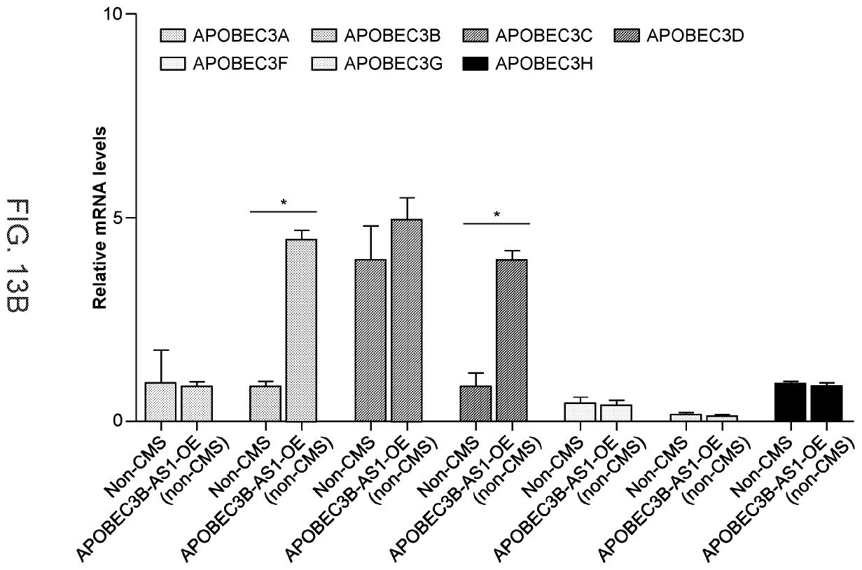

In order to assess the functional roles of APOBEC3B-AS1 and identify its downstream target gene, the chromosomal position and composition of the APOBEC3 gene family were mapped on chromosome 22 as shown in FIG. 13A. Genes from APOBEC3 family that cluster together include APOBEC3A, APOBEC3B, APOBEC3C, APOBEC3D, APOBEC3E. APOBEC3F, APOBEC3G, and APOBEC3H. As shown in FIG. 13B, qPCR results indicated that the expression of APOBEC3B and APOBEC3D was upregulated with OE of APOBEC3B-ASI in non-CMS cells.

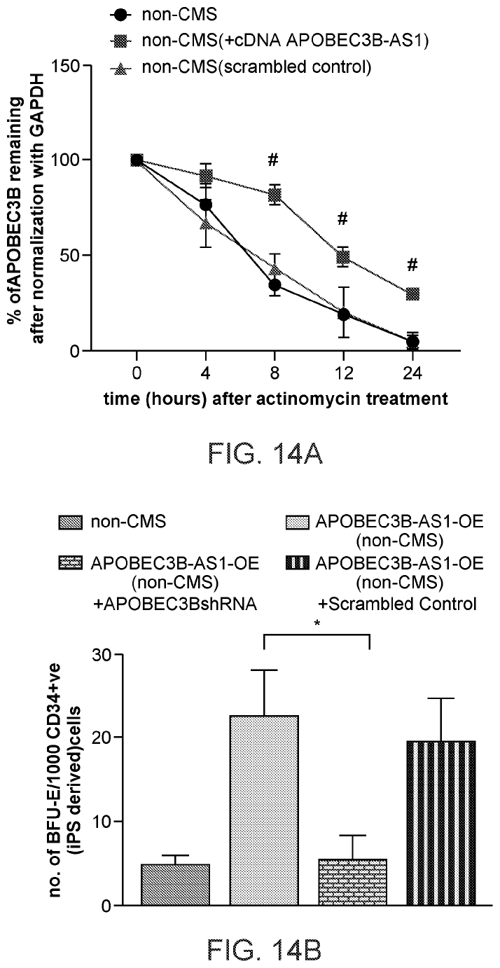

To further assess the functional interactions between APOBEC3B-AS1 and APOBEC3B, mRNA stability assay using transcription inhibition by actinomycin was performed among non-CMS, non-CMS (+cDNA APOBEC3B-AS1), and non-CMS (scrambled control) cells. As shown in FIG. 14A, the OE of APOBEC3B-AS1, achieved by introducing cDNA APOEBC3B-AS1 into non-CMS cells, enhanced stabilization of APOBEC3B under actinomycin treatment. Moreover, FIG. 14B showed that KD of APOBEC3B significantly diminished the erythropoietic response induced by OE of APOBEC3B-AS1, suggesting a functional role of APOBEC3B in mediating the regulation of erythroid cell development by APOBEC3B-AS1.

### D. Csnk2B Inhibitor: Cx-4945

### Example 11—Ongoing Mice Experiments Relevant to this Patent: Testing CX-4945 In Vivo in Mice Under Hypoxia (12% O₂) and Normoxia (21% O₂)

Drastic effect of this drug (CX-4945) has been observed in present in-vitro model in human iPSC-derived erythroid cells from Monge's disease patients and adapted individuals (non-CMS) in terms of BFU-e (erythroid progenitors). In vivo an effect on RBCs has also been obtained in zebrafish embryos model system. But in order to test the efficacy and safety of this drug in vivo, a mouse experiment has recently been started where 12% O₂ was chosen as hypoxia level to mimic high altitude. There were two groups of mice under hypoxia. One group underwent a 3-week acclimatization period under hypoxia before receiving drug treatment, and they remained under hypoxic conditions throughout the treatment. The other group received the drug and was simultaneously exposed to hypoxia without any prior acclimatization. Since the RBCs lifespan in mice is much shorter than humans, this experiment would be critical as a preclinical study to testify whether CX-4945 has an effect on the RBCs levels. Moreover, this experiment could also assess the safety of drug treatment and analyze its potential effects on other cell types, such as white blood cells (WBCs) and platelets, etc. The recently conducted baseline CBC analysis for this experiment and the baseline values for all cell types (RBCs, WBCs, Platelets etc.) demonstrated results within the normal range.

What is claimed is:

1. A method of treating Monge's disease in a subject, the method comprising:
administering to the subject a therapeutic agent, wherein the therapeutic agent inhibits expression of a target gene, and wherein the therapeutic agent reduces erythrocytosis, thereby treating Monge's disease in the subject.

2. A method of reducing erythrocytosis in a subject, the method comprising:
administering to the subject a therapeutic agent, wherein the therapeutic agent inhibits expression of a target gene, thereby reducing erythrocytosis in the subject.

3. The method of claim 1 or 2, wherein the therapeutic agent inhibits expression of the target gene by inhibiting a target RNA, wherein the target RNA regulates the expression of the target gene.

4. The method of claim 3, wherein the target RNA comprises a long non-coding RNA (lncRNA).

5. The method of claim 4, wherein the lncRNA comprises LINC00106, MDC1-AS1, LINC02228, LINC00235, LINC00431, APOBEC3B-AS1, or LINC01133.

6. The method of claim 5, wherein the lncRNA comprises LINC02228.

7. The method of claim 5, wherein the lncRNA comprises LINC00431.

8. The method of claim 5, wherein the lncRNA comprises APOBEC3B-AS1.

9. The method of any one of claims 3-8, wherein the therapeutic agent comprises an inhibitory nucleic acid.

10. The method of claim 9, wherein the inhibitory nucleic acid comprises an antisense oligonucleotide (ASO), a locked nucleic acid (LNA), or a morpholino antisense oligonucleotide.

11. The method of claim 10, wherein the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

12. The method of claim 1 or 2, wherein the target gene comprises CSNK2B, DXO, ZNRD1, PPP1R11, TAP2, IGF1, VCAM1, SHH, TPO, RHEX, SKIV2L, DAXX, ZBTB12, IER3, or any combinations thereof.

13. The method of claim 12, wherein the target gene comprises CSNK2B.

14. The method of claim 13, wherein the therapeutic agent comprises a CK2 inhibitor.

15. The method of claim 14, wherein the therapeutic agent comprises TBB or CX-4945.

16. The method of any one of claims 1-15, wherein the therapeutic agent comprises two or more therapeutic agents.

17. The method of claim 16, wherein the two or more therapeutic agents comprises the inhibitory nucleic acid, the CK2 inhibitor, or any combinations thereof.

18. A pharmaceutical composition comprising a therapeutic agent, wherein the therapeutic agent inhibits expression of a target gene and reduces erythrocytosis.

19. The pharmaceutical composition of claim 18, wherein the therapeutic agent inhibits expression of the target gene by inhibiting a target RNA, wherein the target RNA regulates the expression of the target gene.

20. The pharmaceutical composition of claim 19, wherein the target RNA comprises a long non-coding RNA (lncRNA).

21. The pharmaceutical composition of claim 20, wherein the lncRNA comprises LINC00106, MDC1-AS1, LINC02228, LINC00235, LINC00431, APOBEC3B-AS1, or LINC01133.

22. The pharmaceutical composition of claim 21, wherein the lncRNA comprises LINC02228.

23. The pharmaceutical composition of claim 21, wherein the lncRNA comprises LINC00431.

24. The pharmaceutical composition of claim 21, wherein the lncRNA comprises APOBEC3B-AS1.

25. The pharmaceutical composition of any one of claims 19-24, wherein the therapeutic agent comprises an inhibitory nucleic acid.

26. The pharmaceutical composition of claim 25, wherein the inhibitory nucleic acid comprises an antisense oligonucleotide (ASO), a locked nucleic acid (LNA), or a morpholino antisense oligonucleotide.

27. The pharmaceutical composition of claim 26, wherein the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

28. The pharmaceutical composition of claim 18, wherein the target gene comprises CSNK2B, DXO, ZNRD1, PPP1R11, TAP2, IGF1, VCAM1, SHH, TPO, RHEX, SKIV2L, DAXX, ZBTB12, IER3, or any combinations thereof.

29. The pharmaceutical composition of claim 28, wherein the target gene comprises CSNK2B.

30. The pharmaceutical composition of claim 29, wherein the therapeutic agent comprises a CK2 inhibitor.

31. The pharmaceutical composition of claim 30, wherein the therapeutic agent comprises TBB or CX-4945.

32. The pharmaceutical composition of any one of claims 18-31, wherein the therapeutic agent comprises two or more therapeutic agents.

33. The pharmaceutical composition of claim 32, wherein the two or more therapeutic agents comprise the inhibitory nucleic acid, the CK2 inhibitor, or any combinations thereof.

34. A pharmaceutical composition comprising a therapeutic agent, wherein the therapeutic agent inhibits expression of a target gene and reduces erythrocytosis, for use in the treatment of Monge's disease.

35. The pharmaceutical composition of claim 34, wherein the therapeutic agent inhibits expression of the target gene by inhibiting a target RNA, wherein the target RNA regulates the expression of the target gene.

36. The pharmaceutical composition of claim 35, wherein the target RNA comprises a long non-coding RNA (lncRNA).

37. The pharmaceutical composition of claim 36, wherein the lncRNA comprises LINC00106, MDC1-AS1, LINC02228, LINC00235, LINC00431, APOBEC3B-AS1, or LINC01133.

38. The pharmaceutical composition of claim 37, wherein the lncRNA comprises LINC02228.

39. The pharmaceutical composition of claim 37, wherein the lncRNA comprises LINC00431.

40. The pharmaceutical composition of claim 37, wherein the lncRNA comprises APOBEC3B-AS1.

41. The pharmaceutical composition of any one of claims 35-40, wherein the therapeutic agent comprises an inhibitory nucleic acid.

42. The pharmaceutical composition of claim 41, wherein the inhibitory nucleic acid comprises an antisense oligonucleotide (ASO), a locked nucleic acid (LNA), or a morpholino antisense oligonucleotide.

43. The pharmaceutical composition of claim 42, wherein the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

44. The pharmaceutical composition of claim 34, wherein the target gene comprises CSNK2B, DXO, ZNRD1, PPP1R11, TAP2, IGF1, VCAM1, SHH, TPO, RHEX, SKIV2L, DAXX, ZBTB12, IER3, or any combinations thereof.

45. The pharmaceutical composition of claim 44, wherein the target gene comprises CSNK2B.

46. The pharmaceutical composition of claim 45, wherein the therapeutic agent comprises a CK2 inhibitor.

47. The pharmaceutical composition of claim 46, wherein the therapeutic agent comprises TBB or CX-4945.

48. The pharmaceutical composition of any one of claims 34-47, wherein the therapeutic agent comprises a plurality of therapeutic agents.

49. The pharmaceutical composition of claim 48, wherein the plurality of therapeutic agents comprise the inhibitory nucleic acid, the CK2 inhibitor, or any combinations thereof

50. The use of a pharmaceutical composition comprising a therapeutic agent, in the manufacture of a medicament for treating Monge's disease, wherein the therapeutic agent inhibits expression of a target gene and reduces erythrocytosis.

51. The use of the pharmaceutical composition of claim 50, wherein the therapeutic agent inhibits expression of the target gene by inhibiting a target RNA, wherein the target RNA regulates the expression of the target gene.

52. The use of the pharmaceutical composition of claim 51, wherein the target RNA comprises a long non-coding RNA (lncRNA).

53. The use of the pharmaceutical composition of claim 52, wherein the lncRNA comprises LINC00106, MDC1-AS1, LINC02228, LINC00235, LINC00431, APOBEC3B-AS1, or LINC01133.

54. The use of the pharmaceutical composition of claim 53, wherein the lncRNA comprises LINC02228.

55. The use of the pharmaceutical composition of claim 53, wherein the lncRNA comprises LINC00431.

56. The use of the pharmaceutical composition of claim 53, wherein the lncRNA comprises APOBEC3B-AS1.

57. The use of the pharmaceutical composition of any one of claims 51-56, wherein the therapeutic agent comprises an inhibitory nucleic acid.

58. The use of the pharmaceutical composition of claim 57, wherein the inhibitory nucleic acid comprises an antisense oligonucleotide (ASO), a locked nucleic acid (LNA), or a morpholino antisense oligonucleotide.

59. The use of the pharmaceutical composition of claim 58, wherein the inhibitory nucleic acid comprises SEQ ID NO: 1, SEQ ID NO: 2, SEQ ID NO: 3, SEQ ID NO: 4, SEQ ID NO: 5, SEQ ID NO: 6, SEQ ID NO: 7, or any combinations thereof.

60. The use of the pharmaceutical composition of claim 50, wherein the target gene comprises CSNK2B, DXO, ZNRD1, PPP1R1l, TAP2, IGF1, VCAM1, SHH, TPO, RHEX, SKIV2L, DAXX, ZBTB12, IER3, or any combinations thereof.

61. The use of the pharmaceutical composition of claim 50 or 60, wherein the target gene comprises CSNK2B.

62. The use of the pharmaceutical composition of claims 50, 60-61, wherein the therapeutic agent comprises a CK2 inhibitor.

63. The use of the pharmaceutical composition of claims 50, 60-62, wherein the therapeutic agent comprises TBB or CX-4945.

64. The use of the pharmaceutical composition of any one of claims 50-63, wherein the therapeutic agent comprises a plurality of therapeutic agents.

65. The use of the pharmaceutical composition of claim 64, wherein the plurality of therapeutic agents comprise the inhibitory nucleic acid, the CK2 inhibitor, or any combinations thereof.


SEQUENCE LISTING (25 sequences)

SEQ ID NO: 1 - DNA, 20 bp, synthetic construct
  source 1..20: mol_type = other DNA
ggttgctttcaggcccctta

SEQ ID NO: 2 - DNA, 20 bp, synthetic construct
  source 1..20: mol_type = other DNA
cttcaccatgcttgtgctct

SEQ ID NO: 3 - DNA, 20 bp, synthetic construct
  source 1..20: mol_type = other DNA
gccttccgggccgtccacgc

SEQ ID NO: 4 - DNA, 20 bp, synthetic construct
  source 1..20: mol_type = other DNA
tttcctcggtccatgcacac

SEQ ID NO: 5 - DNA, 20 bp, synthetic construct
  source 1..20: mol_type = other DNA
acgatacccttgctcccaac

SEQ ID NO: 6 - DNA, 20 bp, synthetic construct
  source 1..20: mol_type = other DNA
accggctcccatggtgtctt

SEQ ID NO: 7 - DNA, 21 bp, synthetic construct
  source 1..21: mol_type = other DNA
ggagcgagatccctccaaaat

SEQ ID NO: 8 - DNA, 23 bp, synthetic construct
  source 1..23: mol_type = other DNA
ggctgttgtcatacttctcatgg

SEQ ID NO: 9 - DNA, 20 bp, synthetic construct
  source 1..20: mol_type = other DNA
ccacatcgctcagacaccat

SEQ ID NO: 10 - DNA, 17 bp, synthetic construct
  source 1..17: mol_type = other DNA
accaggcgcccaatacg

SEQ ID NO: 11 - DNA, 21 bp, synthetic construct
  source 1..21: mol_type = other DNA
tcccagatgtgccaaagtcag

SEQ ID NO: 12 - DNA, 20 bp, synthetic construct
  source 1..20: mol_type = other DNA
agcaaccccagttgtcattc

SEQ ID NO: 13 - DNA, 24 bp, synthetic construct
  source 1..24: mol_type = other DNA
ggaacatgtttacatggactttgt

SEQ ID NO: 14 - DNA, 26 bp, synthetic construct
  source 1..26: mol_type = other DNA
tctttgttcttgtttgttttctttct

SEQ ID NO: 15 - DNA, 20 bp, synthetic construct
  source 1..20: mol_type = other DNA
ctgaaagcaacctccagtcc

SEQ ID NO: 16 - DNA, 20 bp, synthetic construct
  source 1..20: mol_type = other DNA
gccagttgcatattgcttca

SEQ ID NO: 17 - DNA, 20 bp, synthetic construct
  source 1..20: mol_type = other DNA
ctcttactgctgggcctgtc

SEQ ID NO: 18 - DNA, 20 bp, synthetic construct
  source 1..20: mol_type = other DNA
agaccgttcaccaccatctc

SEQ ID NO: 19 - DNA, 20 bp, synthetic construct
  source 1..20: mol_type = other DNA
cattgctgaagttggactcg

SEQ ID NO: 20 - DNA, 20 bp, synthetic construct
  source 1..20: mol_type = other DNA
tctcgcaggctaaactgacc

SEQ ID NO: 21 - DNA, 20 bp, synthetic construct
  source 1..20: mol_type = other DNA
gtttcctccctcctcaatcc

SEQ ID NO: 22 - DNA, 20 bp, synthetic construct
  source 1..20: mol_type = other DNA
cctctgcagacgtgtattcg

SEQ ID NO: 23 - DNA, 25 bp, synthetic construct
  source 1..25: mol_type = other DNA
cgacacttcctctgagctactcatg

SEQ ID NO: 24 - DNA, 25 bp, synthetic construct
  source 1..25: mol_type = other DNA
cgagagttcgtctgacctagtcatg

SEQ ID NO: 25 - DNA, 24 bp, synthetic construct
  source 1..24: mol_type = other DNA
atgagtagctcagaagaggtctcc